(19) United States
(12) Patent Application Publication
(10) Pub. No.: US 2009/0286304 A1
(43) Pub. Date: Nov. 19, 2009

(54) ENZYMATIC DIGESTION OF TISSUE

(75) Inventors: Gary Latham, Austin, TX (US); Brittan L. Pasloske, Austin, TX (US); Heidi Peltier, Austin, TX (US)

Correspondence Address: LIFE TECHNOLOGIES CORPORATION;C/O INTELLEVATE, P.O. BOX 52050, MINNEAPOLIS, MN, 55402, US

(73) Assignee: LIFE TECHNOLOGIES CORPORATION, Carlsbad, CA (US)

(21) Appl. No.: 12/463,780

(22) Filed: May 11, 2009

Related U.S. Application Data
(63) Continuation of application No. 11/076,455, filed on Mar. 9, 2005, now abandoned.
(60) Provisional application No. 60/654,219, filed on Feb. 18, 2005.

(51) Int. Cl. C12N 1/08

(52) U.S. Cl. 435/270

(57) ABSTRACT

The present invention concerns a compositions and method for isolating a nucleic acid from a cell-containing sample. There is disclosed a method comprising obtaining at least one cell-containing sample, which comprises a cell containing nucleic acid, obtaining at least one catabolic enzyme, obtaining at least one nuclease inhibitor, preparing an admixture of the sample, the catabolic enzyme, and the nuclease inhibitor, maintaining the admixture under conditions where the catabolic enzyme is active, and agitating the admixture, where the sample is digested to produce a nucleic acid-containing lysate of the sample.

This application is a continuation application of pending U.S. patent application Ser. No. 11/076,455 filed Mar. 9, 2005, which application claims the benefit of U.S. Provisional Application No. 60/654,219, filed Feb. 18, 2005. The contents of the cited applications are incorporated by reference herein.

### BACKGROUND OF THE INVENTION

1. Field of the Invention

The present invention relates generally to the field of molecular biology. More particularly, it concerns methods and compositions for isolating and preserving nucleic acid such as RNA of high quality and yield from tissue.

2. Description of Related Art

Tissue samples are invaluable for understanding, diagnosing, and treating a disease. In both research and clinical settings, diseased and normal tissues provide genomic and proteomic profiles that “fingerprint” their biological status. These profiles can be correlated with specific patterns of gene expression that link specific molecular events with the disease phenotype.

Current approaches to gene expression analysis typically hinge on the isolation of intact, high-quality RNA, which can serve as a “snapshot” of the expression profile. The first step of most RNA extractions is to rupture the source tissue with a dounce, polytron, mill, or other device for mechanical disruption of the tissue. These methods are cumbersome, provide low throughput, requiring washing of the disruption apparatus between samples and are potentially inefficient. Such methods can also present a biological hazard by exposing the operator to aerosols from the diseased samples.

High throughput isolation of nucleic acid from tissues, human biopsies, animal bio-samples, and plants, for example, represent an emerging need that is currently not met by the prior art.

### SUMMARY OF THE INVENTION

The present invention overcomes the deficiencies in the art by providing compositions and methods for their use that can be used to preserve and/or isolate nucleic acid such as RNA or DNA from a cell-containing sample or a biological unit.

In one aspect of the invention, for example, there is provided a method comprising obtaining at least one cell-containing sample or biological unit, which comprises a cell containing nucleic acid, obtaining at least one catabolic enzyme, obtaining at least one nuclease inhibitor, preparing an admixture of the sample, the catabolic enzyme, and the nuclease inhibitor, and maintaining the admixture under conditions where the catabolic enzyme is active, and agitating the admixture, wherein the sample is digested to produce a nucleic acid-containing lysate of the sample. Digestion means a process in which the cellular or extracellular architecture is degraded. Digestion, in certain aspects, can occur with or without contacting the cell-containing sample with a mechanical object. It is contemplated, however, that during agitation, the cell-containing sample may come in contact with the container or tube that the admixture is in. In other aspects, the digestion can occur without homogenizing the cell-containing sample. Homogenizing occurs by using a homogenizer such as a: (1) Polytron® and/or rotor stator homogenizer (such as the Tissue Tearor); (2) dounce; (3) mortar and pestle; (4) tissue mill, mixer-mill, or bead-beater assembly (e.g., adding metal beads to tube with lysis buffer and the tissue sample and shaking), examples include TissueLyser (Qiagen) and the mini-beadbeater-8 (Biospec); (5) blender, such as a Waring® Blender; (6) spin column homogenizer, such as the QIAshredder (Qiagen); and (7) sonicator, such as the Cole-Parmer® 130-Watt Ultrasonic Processor.

A person of ordinary skill in the art will recognize that many types of catabolic enzyme can be useful with the present invention. Non-limiting examples of catabolic enzymes include enzymes that can degrade proteins, carbohydrates, lipids, DNA, RNA, and other cellular and non-cellular molecules. Specific non-limiting examples include proteases, collagenases, elastases, hyaluronidases, trypsins, chymotrypsins, papain, proteinase K, lipases, DNases (e.g., exonucleases and endonucleases, including but not limited to DNase I, DNase II, and Shrimp arctic DNase), RNases, amylases, cellulases, and other catabolic enzymes discussed throughout this specification such as the enzymes listed in Tables 2 and 3A and 3B which are incorporated into this section by reference. In certain aspects, the catabolic enzyme is a protease. The protease can be, for example, Proteinase K, which is recognized to be a member of the broad family of Subtilisin-like enzymes. The protease may also be Subtilisin, of which a number of enzyme subtypes exist, including, for example: (1) B. amyloliquefaciens Subtilisin also known as Subtilisin BPN, Subtilisin novo, BAS, Neutrase, bacterial protease Novo, furilysin, Nagarse, subtilopeptidase B, subtilopeptidase C, or Subtilisin B; (2) B. licheniformis Subtilisin also known as Subtilisin Carlsberg, Alcalase, Maxatase, Versazyme™ Keratinase (strain PWD-1) from BioResource International, Inc., Subtilisin A, or thiosubtilisin; (3) B. licheniformis engineered Subtilisin, Purafect™, Purafect Ox™, or Properase™ from Genencor; (4) B. lentus Subtilisin also known as Savinase, Everlase a protein-engineered variant of Savinase®, Esperase, Maxacal, protease PB92 (Bacillus sp.), Subtilisin 309, or Subtilisin BL; (5) B. subtilis Subtilisin also known as Subtilisin E′ or Subtilisin 147. It is also contemplated that any engineered Subtilisin (e.g., chemical or molecular engineered) or derivatives of the Subtilisins discussed above and throughout this document can be used with the present invention. In other aspects, the protease can be a cysteine protease (e.g., papain), or a keratinase, for example. These and other proteases discussed throughout this document and known to those of ordinary skill in the art are also contemplated as being useful with the present invention.

In particular aspects, the method comprises obtaining at least two, three, four, five, six, seven, eight, nine, ten, eleven, twelve, thirteen, fourteen, or more catabolic enzymes. The catabolic enzymes can be admixed together in any number of combinations. For example, Tables 2 and 3A and 3B provide non-limiting examples of certain admixtures of catabolic enzymes. The catabolic enzymes can be admixed together with or without the nuclease inhibitor.

In non-limiting aspects, the concentration range of a given catabolic enzyme can be, for example, between about 0.001 mg/ml to about 50 mg/ml. In other embodiments the range can be between from about 0.01 mg/ml to about 5 mg/ml, or between about 0.2 mg/ml to about 1.0 mg/ml. In particular aspects the concentration can be about 0.4 mg/ml. Of course, it should be recognized that two or more catabolic enzymes may be used at the same or different amounts. It is further contemplated that two or more enzymes may be used at concentrations below or above these concentration ranges because of, for example, a synergistic effect between the two or more enzymes, therefore, reducing the amount of enzyme needed for a given assay or procedure.

In certain preferred embodiments, for example, the catabolic enzyme is Proteinase K or Subtilisin Carlsberg, or both. These enzymes can be provided, for example, at a stock concentration range of from about 1 mg/ml to about 50 mg/ml each. In other aspects where the enzymes are included in a kit, the stock concentration range can be from about 10 mg/ml to about 30 mg/ml each, or from about 15 mg/ml to about 25 mg/ml each. In some particular aspects, the stock concentration of the enzymes can be about 20 mg/ml each. The concentration of these enzymes in the lysate can range, for example, from about 0.001 mg/ml to about 25 mg/ml each, from 0.1 mg/ml to 10 mg/ml each, or from 0.2 mg/ml to 0.6 mg/ml each. In certain aspects, the concentration of these enzymes in the lysate can be about 0.4 mg/ml each.

Non-limiting examples of nuclease inhibitors that can be used with the present invention include, for example, RNase inhibitors (e.g., detergents, small molecules, antibodies, and proteinaceous and non-proteinaceous compounds) and all other nuclease inhibitors that are discussed throughout this document and known to those of skill in the art which are incorporated into this section by reference. The methods can include, for example, obtaining at least two, three, four, five, six, seven, eight, nine, ten, eleven, twelve, thirteen, fourteen, or more RNase inhibitors. The RNase inhibitors can be admixed together in any number of combinations.

The concentration range of the nuclease inhibitors in the lysate can vary depending on the nature of the nuclease inhibitor that is used. For example, if the nuclease inhibitor is a detergent, the concentration range can be, in non-limiting aspects, from about 0.1 to about 10% (w/v). In other aspects, the concentration range can be from about 0.5% to 5%, or from about 1% to about 3%. In certain aspects, the concentration range is about 2%. If the nuclease inhibitor is a small molecule, the concentration range in the lysate can be from about 0.001 mM to 5M, from 0.01 mM to 500 mM, or from 0.1 mM to 50 mM, for example. If the nuclease inhibitor is an antibody, for example, the concentration range can be from about 0.01 μg/ml to 50 mg/ml, from about 0.1 μg/ml to about 5 mg/ml, or from about 1 μg/ml to about 500 μg/ml. If the nuclease inhibitor is a proteinaceous compound, for example, the concentration range can be from about 0.01 pM to about 1 mM, from about 1 pM to about 100 uM, or from about 10 pM to about 1 uM. If the nuclease inhibitor is a non-proteinaceous compound, for example, the concentration range can be from about 0.001 mM to about 5M, from about 0.01 mM to about 500 mM, or from about 0.1 mM to about 50 mM. The stock concentrations provided in a kit, in non-limiting embodiments, can be about 2 times to about 100 times the final concentration in the lysate. For example, in other non-limiting aspects, the kit can include sodium dodecyl sulfate (SDS). The concentration of SDS in a kit, for example, can be from about 0.001% to about 90% w/v, from about 0.01% to about 50% w/v, or from about 0.1% to about 10% w/v of SDS.

Non-limiting examples of detergents include ionic (e.g. cationic, anionic, zwitterionic) or non-ionic detergents or mixtures thereof. In certain aspects, the ionic detergent is an anionic detergent. The anionic detergent can be, for example, a dodecyl or lauryl sulfate detergent (e.g., sodium dodecyl sulfate, sodium lauryl sulfate, lithium dodecyl sulfate, lithium lauryl sulfate, trizma® dodecyl sulfate), N-lauryl sarcosine, chenodeoxycholic acid, cholic acid, dehydrocholic acid, deoxycholic acid, digitonin, digitoxigenin, N,N-Dimethyldodecylamine N-oxide, docusate, glycochenodeoxycholic acid, glycocholic acid, glycodeoxycholic acid, glycolithocholic acid ethyl ester, N-Lauroylsarcosine, lugol solution, niaproof 4,1-Octanesulfonic acid, sodium 1-butanesulfonate, sodium 1-decanesulfonate, sodium 1-dodecanesulfonate, sodium 1-heptanesulfonate, sodium 1-nonanesulfonate, sodium 1-propanesulfonate, sodium 2-bromoethanesulfonate, sodium choleate, sodium deoxycholate, sodium hexanesulfonate anhydrous, sodium octyl sulfate, sodium pentanesulfonate anhydrous, sodium taurocholate, taurochenodeoxycholic acid sodium salt, taurohyodeoxycholic acid sodium salt hydrate, taurolithocholic acid 3-sulfate disodium salt, tauroursodeoxycholic acid sodium salt, ursodeoxycholic acid, or triisopropylnaphthalene sulphonic acid/p-aminosalicylic acid, or combination thereof.

Non-proteinaceous compounds can be, for example, small molecules, BpB, BpB analogs, ADP, or a vanadyl complex. Small molecule inhibitors can include, for example, compounds that include an aromatic structure or a polycyclic aromatic structure, or both. Examples of proteinaceous compounds include placental ribonuclease inhibitors or anti-RNase antibodies.

The isolated nucleic acid can be preserved intact in the lysate. “Intact” can be described in functional terms as a condition where the isolated nucleic acid is in a form that is sufficient to be used in a relevant procedure. Non-limiting examples of relevant procedures include RNA or DNA amplification reactions, RNA or DNA labeling reactions, RNA or DNA isolation reactions, RNA or DNA digestion reactions, in vitro translation reactions, in vitro transcription reactions, reverse transcription reactions, in vitro coupled transcription/translation reactions, or any nucleic acid based detection method that is based on hybridization (e.g., southern blotting, microarray detection, northern blotting, or ribonuclease protection assays). In other aspects, “intact” RNA includes a 28S/18S ratio of about greater than or equal to about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0. In some cases, intactness will not be a long term consideration. This can occur in situations, for example, where after the treatment or digestion of the cell-containing sample, the RNA will be immediately, or soon thereafter, isolated or otherwise used for its intended purpose.

During digestion, the admixture can be maintained at a temperature where the catabolic enzyme is active and the RNA is preserved intact. In non-limiting aspects, the temperature can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, or more degrees celsius. Non limiting ranges include between about 4° C. and about 70° C., between about 20° C. and about 55° C., or between about 30° C. and about 40° C. In other embodiments, the admixture is maintained at room temperature. “Room temperature” means maintaining the admixture at the ambient temperature of a given room (e.g., a lab), and in most normal cases, this would encompass a temperature of about 20° C. to about 25° C. Also contemplated is the use of temperature ramping (i.e., increasing or decreasing the temperature from a start point to an end point) and/or using multiple temperatures for a given protocol or assay.

During storage, the nucleic acid-containing lysate can be stored at a variety of temperatures. Non-limiting temperatures include −100, −90, −80, −70, −60, −50, −40, −30, −20, −10, −9, −8, −7, −6, −5, −4, −3, −2, −1, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39,40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 86, 86, 87, 88, 89, 90, 91, 92, 93, 94, 95, 96, 97, 98, 99, 100, or more degree celsius. Non limiting ranges include between about 4° C. and about 70° C., between about 10° C. and about 40° C., or between about 20° C. and about 30° C. In other embodiments, the nucleic acid-containing lysate can be stored at room temperature.

In certain non-limiting embodiments, the RNA can be preserved intact for at least 1, 2, 3, 4, 5, 6, 7, 8, 9, 10, 11, 12, 13, 14, 15, 20, 30, 40, 50, 60, or 90 minutes or more. In other aspects, the RNA can be preserved intact for at least 2, 3, 4, 5, 6, 7, 8, 9, 10, 11, 12, 13, 14, 15, 16, 17, 18, 19, 20, 21, 22, 23, or 24 hours or more. In other aspects, RNA preservation can last for at least 1, 2, 3, 4, 5, 6, 7, 8, 9 10, 20, 30, 40, 50, 60, 70, 100, 150, 200, 300, or 365 days or indefinitely.

Non-limiting examples of agitation include shaking, stirring, mixing, or vibrating the admixture. In certain aspects, agitation includes shaking. The shaking can be one, two, or three dimensional shaking. A variety of shaking or agitating devices can be used. Non-limiting examples include the Thermomixer (Eppendorf), TurboMix (Scientific Industries), Mo Bio Vortex Adapter (Mo Bio Laboratories), Microtube holder vortex adapter (Troemner), and the Microtube foam rack vortex attachment (Scientific Industries). In certain aspects, however, three-dimensional shaking is preferred.

In certain non-limiting embodiments, the digestion of the sample occurs within about 1, 2, 3, 4, 5, 6, 7, 8, 9, 10, 11, 12, 13, 14, 15, 16, 17, 18, 19, 20, 25, 30, 35, 40, 45, 50, 55, 60, or about 90 minutes or less. Digestion can also occur within about 2, 3, 4, 5, 6, 7, 8, 9, 10, 15, 20, or 24 hours or 1, 2, 3, 4, or 5, days or less. While one aspect and advantage of the invention is that it can allow for rapid digestion, not all embodiments of the invention require such rapid digestion, and methods and compositions where digestion takes hours or even days can have advantages or be useful in some applications.

A cell-containing sample can include, in a non-limiting embodiment, a tissue sample. In certain aspects, a tissue sample includes any collection of two or more cells that are isolated from a subject. A subject includes any organism from which a sample can be isolated. Non-limiting examples of organisms include eukaryotes such as fungi, animals, plants, or protists. The animal, for example, can be a mammal or a non-mammal. The mammal can be, for example, a mouse, rat, rabbit, dog, pig, cow, horse, rodent, or a human. In particular aspects, the tissue sample is a human tissue sample. The tissue sample can be, for example, a blood sample. The blood sample can be blood (e.g., red blood cells, white blood cells, platelets, plasma, serum, or whole blood). The sample, in other non-limiting embodiments, can be saliva, a cheek, throat, or nasal swab, a fine needle aspirate, a tissue print, cerebral spinal fluid, mucus, semen, lymph, feces, or urine. In other aspects, the tissue sample is a solid tissue sample. Other tissue samples that are described throughout this document are contemplated as being useful with the present invention and are incorporated into this section by reference.

In still other aspects, the tissue sample may comprise a biological unit. The biological unit, in non-limiting aspect, can include a virus, bacteria, or fungus. The term “biological unit” is defined to mean any cell, virus, fungus, or bacteria that contains genetic material. In most aspects of the invention, the genetic material of the biological unit will include RNA. In some embodiments, the biological unit is a prokaryotic or eukaryotic cell, for example a bacterial, fungal, plant, protist, animal, invertebrate, vertebrate, mammalian, rodent, mouse, rat, hamster, primate, or human cell. Such cells may be obtained from any source possible, as will be understood by those of skill in the art. For example, a prokaryotic or eukaryotic cell culture. The biological unit may also be obtained from a sample from a subject or the environment. The subject may be an animal, including a human. The biological can also be from a body fluid, e.g., whole blood, plasma, serum, urine or cerebral spinal fluid.

It is also contemplated that the methods and compositions of the present invention is applicable with cell-free samples that contain nucleic acid. Non-limiting examples of cell-free samples include plasma, serum, saliva, urine, and cerebral spinal fluid (CSF), and other cell free samples that are discussed in this document and known to those of ordinary skill in the art.

The method can be further defined as a method of inactivating ribonucleases in the lysate. The method can further include isolating the nucleic acid (e.g., RNA or DNA) from the lysate. The isolation can include, in certain aspects, binding the nucleic acid to a magnetic bead. In other embodiments, the isolation comprises employing a filter-based technique.

The method can be further defined as a method for producing cDNA from RNA in the lysate. This can be performed by incorporating a reverse transcription. The method can also include amplifying products of the reverse transcription. In other aspects, the method can further include hybridizing nucleic acid from the lysate to another nucleic acid.

The present methods, compounds, reagents, and kits disclosed in this specification can be used in several biological techniques. Non-limiting examples include RNA or DNA amplification reactions, RNA or DNA labeling reactions, RNA or DNA isolation reactions, RNA or DNA digestion reactions, in vitro translation reactions, in vitro transcription reactions, reverse transcription reactions, in vitro coupled transcription/translation reactions, or any nucleic acid based detection method that is based on hybridization (e.g., southern blotting, microarray detection, northern blotting, or hybridization protection or ribonuclease protection assays). Other molecular biology techniques that are known to those of skill in the art are also contemplated as being useful with all aspects of the present invention.

In another embodiment of the present invention, there is disclosed a kit that can be used to preserve and isolate nucleic acid such as RNA or DNA. The kit can also be used for producing a lysate of a tissue sample, comprising, in a suitable container, a buffer, a catabolic enzyme, and a nuclease inhibitor. The kit can further include salts (e.g., NaCl, KCl, MgCl₂, or CaCl₂), water (e.g., nuclease-free water), nucleic acid binding beads (e.g., RNA binding beads), RNA binding solutions, RNA elution solutions, or washing solutions. The buffer, for example, can have a variety of pH ranges including 1, 2, 3, 4, 5, 6, 7, 8, 9, 10, 11, or 12, or more. In certain embodiments, the buffer is a buffer that includes CHES, CaCl₂, EDTA and SDS. The buffer can be a DNase 1 buffer that includes, for example, Tris, MgCl₂ or CaCl₂. The RNA binding solution can include NaCl, Tris-HCl, and β-mercaptoethanol. The RNA elution solution can include, for example, NaCl and EDTA. The washing solution can include KCl, Tris-HCl, EDTA, and ethanol. In particular embodiments, the kit may also contain one or more catabolic enzyme cocktails. For example, one cocktail can include Proteinase K and a storage buffer which can include Tris, CaCl₂, and Glycerol. In another example, the cocktail can include Subtilisin Carlsberg and a storage buffer, the storage buffer including Tris, CaCl₂, and Glycerol. These and other aspects of the kit are disclosed throughout this documents and are incorporated into this section be reference.

The inventors also contemplate a sample lysis digestion solution. The digestion solution, for example, can be used to digest, preserve, and isolate intact nucleic acid (e.g., RNA or DNA). The sample lysis buffer can be used at a variety of temperatures, including temperatures that allow its ingredients (e.g., catabolic enzymes and nuclease inhibitor) to remain active such as room temperature. Other non-limiting temperatures include, for example, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80 degrees celsius, or greater. The obtained nucleic acid containing lysate can be preserved at a variety of temperatures including, but not limited to room temperature, −100, −90, −80, −70, −60, −50, −40, −30, −20, −10, −9, −8, −7, −6, −5, −4, −3, −2, −1, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37,38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 86, 86, 87, 88, 89, 90, 91, 92, 93, 94, 95, 96, 97, 98, 99, 100, or more degree celsius. Preservation of the nucleic acid containing lysate can also occur over an extended period of time (e.g., more than 1, 2, 3, 4, 5, 6, 7, 8, 9, 10, 15, 20, 25, 30, 35, 40, 45, 50, 55, 60, or 90 minutes or more than 2, 3, 4, 5, 6, 7, 8, 9, 10, 15, 20, or 24 hours, or more than 2, 3, 4, 5, 6, 7, or more days.). The sample lysis digestion solution can also be used to digest a cell-containing sample in short periods of time (e.g., less than 1, 2, 3, 4, 5, 6, 7, 8, 9, 10, 15, 20, 25, 30, 35, 40, 45, 50, 55, 60, or 90 minutes). The sample lysis digestion solution can include at least one cell-containing sample, which comprises a cell-containing nucleic acid; at least one catabolic enzyme; at least one nuclease inhibitor; and a buffer, wherein the buffer includes a pH range of between about 7 and about 10, and wherein the buffer is formulated to maintain the activity of the catabolic enzyme and the nuclease inhibitor. Of course, the pH range can vary below 7 (including 6, 5, 4, 3, 2, and 1) and above 7 (including 8, 9, 10, 11, 12, 13, or 14) depending on, for example, the components in the sample lysis digestion solution. The at least one catabolic enzyme in the sample lysis digestion solution can be Proteinase K or any other catabolic enzyme. The buffer can further include any second catabolic enzyme. The second catabolic enzyme, in certain aspects, can be Subtilisin. The Subtilisin can be, for example, Subtilisin Carlsberg. The buffer can include any third catabolic enzyme. The third catabolic enzyme can be DNase 1. The sample lysis buffer can include, in one aspect, from about 0.001 to about 10 mg/ml of the catabolic enzyme, from about 0.1 to about 1 mg/ml of the catabolic enzyme, or about 0.4 mg/ml of the catabolic enzyme. In certain embodiments, the nuclease inhibitor is an RNase inhibitor. The RNase inhibitor can be, for example, SDS or any other inhibitors discussed throughout this document. The sample lysis buffer can include from about 0.1% to about 10%, from about 0.5% to about 5%, to about 2% of the RNase inhibitor when the inhibitor is an anionic detergent. In other aspects, the buffer includes CHES, CaCl₂, EDTA, or SDS.

In still another aspect of the current invention, there is disclosed a method of preserving RNA in a tissue lysate comprising obtaining at least one tissue sample, which comprises cells containing RNA, obtaining at least one catabolic enzyme, obtaining at least one ribonuclease inhibitor, preparing an admixture of the sample, the catabolic enzyme, and the ribonuclease inhibitor, maintaining the admixture under conditions where the catabolic enzyme is active, and agitating the admixture, where the sample is digested to produce an RNA containing lysate in which the RNA is preserved. In certain non-limiting aspects, preserved is further defined as RNA including a 28S/18S ratio of about 0.5, after about 3 days at 22-25° C. In other aspects, the 28S/18S ratio is about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0. Further the preservation temperature and length of preservation can vary. For example, preservation temperatures can include in non-limiting aspects room temperature, −100, −90, −80, −70, −60, −50, −40, −30, −20, −10, −9, −8, −7, −6, −5, −4, −3, −2, −1, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 86, 86, 87, 88, 89, 90, 91, 92, 93, 94, 95, 96, 97, 98, 99, 100, or more degree celsius. The length of preservation can include in non-limiting embodiments more than 1, 2, 3, 4, 5, 6, 7, 8, 9, 10, 15, 20, 25, 30, 35, 40, 45, 50, 55, 60, or 90 minutes or more than 2, 3, 4, 5, 6, 7, 8, 9, 10, 15, 20, or 24 hours, or more than 2, 3, 4, 5, 6, 7 days, or more than 1, 2, 3, 4, 5, 6, 7 years or more. It should therefore be recognized, that preservation standards can vary depending on, for example, the parameters of any given assay or the desired RNA quality that the user would like to achieve. In certain embodiments, RNA is preserved intact. Digestion can occur without homogenizing the cell-containing sample. The RNA lysate can be preserved at pH of about 1, 2, 3, 4, 5, 6, 7, 8, 9, 10, 11, 12, or more. In preferred embodiments, it is preserved at a pH of about 7 to about 10.

It is contemplated that any method or composition described herein can be implemented with respect to any other method or composition described herein.

The methods and compositions of the present invention can be performed in a closed or open system. Additionally, the methods of the present invention can be performed in one, two, three, four, or more separate containers or tubes. For example, lysis of the cell-containing sample may occur in one container along with a biological procedure. The biological procedure can be for example, reverse transcription reactions, RNA or DNA amplification reactions, RNA or DNA labeling reactions, RNA or DNA isolation reactions, RNA or DNA digestion reactions, in vitro translation reactions, in vitro transcription reactions, in vitro coupled transcription/translation reactions, or any nucleic acid based detection method that is based on hybridization (e.g., southern blotting, microarray detection, northern blotting, or ribonuclease protection assays). In other aspects, multiple containers may be used. For example, lysis can occur in one container and a RT-PCR reaction can take place in a second container.

As used herein, the terms “cell,” “cell line,” and “cell culture” may be used interchangeably. All of these terms also include their progeny, which is any and all subsequent generations. Cells may be derived from prokaryotes or eukaryotes. In certain embodiments, a cell may comprise, but is not limited to, at least one skin, bone, neuron, axon, cartilage, blood vessel, cornea, muscle, facia, brain, prostate, breast, endometrium, lung, pancreas, small intestine, blood, liver, testes, ovaries, cervix, colon, skin, stomach, esophagus, spleen, lymph node, bone marrow, kidney, peripheral blood, embryonic or ascite cell, and all cancers thereof.

The compositions and methods of the present invention are compatible with all types of tissues, including but not limited to, fresh, flash-frozen, fixed, RNAlater® or RNAlater®-ICE (Ambion, Inc., Austin, Tex.) preserved tissue.

The terms “nuclease inactivation” or the “inactivation of nucleases” connotes that there is no detectable degradation of the sample DNA or RNA under the assay conditions used, and that the nuclease is irreversibly rendered inoperative.

The term “substantially inactivated” connotes that there is no substantial degradation of DNA or RNA detected in a composition that may contain DNA or RNA, and that a measurable loss in the nuclease results from irreversible inactivation, whereby the nuclease is rendered inoperative.

The terms “inhibiting,” “reducing,” or “prevention,” or any variation of these terms, when used in the claims and/or the specification includes any measurable decrease or complete inhibition to achieve a desired result. “Inhibiting” does not require complete nuclease inactivation or even substantial nuclease inactivation. The term “substantial inhibition” connotes that there is no substantial degradation of DNA or RNA detected in a composition that may include DNA or RNA.

Throughout this application, the term “about” is used to indicate that a value includes the standard deviation of error for the device or method being employed to determine the value.

The use of the word “a” or “an” when used in conjunction with the term “comprising” in the claims and/or the specification may mean “one,” but it is also consistent with the meaning of “one or more,” “at least one,” and “one or more than one.”

It is specifically contemplated that any embodiments described in the Examples section are included as an embodiment of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A and FIG. 1B: Correlation of Normalized Array Signal Intensities. Total RNA was isolated from fresh mouse brain (A) and liver (B) tissues (˜7 mg of tissue processed/reaction). A signal correlation plot showed 0.99 correlation between normalized microarray data from samples prepared using two isolation methods using the (I) Enzymatic Lysis of Tissue (ELT) System and (2) Affymetrix-recommended RNA isolation procedure (TRI® Reagent followed by glass-filter purification).

FIG. 2: Total RNA profile demonstrating intact RNA from a variety of tissue types.

FIG. 3: RNA preservation in tissue lysates over days at room temperature. ELT is Enzymatic Lysis of Tissue (ELT), one aspect of the present invention. GuSCN is Guanidine Thiocyanate.

### DESCRIPTION OF ILLUSTRATIVE EMBODIMENTS

Nucleic acids such as RNA or DNA are invaluable for understanding, diagnosing, and treating diseases, solving crimes, and discovering new cures to old diseases. In order to use nucleic acids, it is usually necessary to isolate the nucleic acids for subsequent analysis or amplification.

To overcome the problems inherent with previous nucleic acid isolation systems, the inventors have developed compositions and methods for their use that can, for example, be used to obtain intact nucleic acid from a variety of cell-containing samples in a relatively short period of time and in a simple and efficient manner. In certain aspects, the invention allows researchers to disrupt intact tissue samples without the need for a polytron, mortar and pestle, or other physical grinding method that is tedious, low throughput, involves washing of the disruption apparatus between each sample and vulnerable to cross-sample contamination. The compositions and methods described herein allow for faster sample processing times, improved nucleic acid yields, ready automation, and reduced variability, contamination, and biohazard risks. The obtained nucleic acid can be preserved at a variety of temperatures and for extended periods of time. Additionally, the preservation of the nucleic acid is significantly better than that achievable by other known methods. Currently, secure methods for preserving RNA in tissue samples prior to sample disruption are to flash-freeze the intact tissue in liquid nitrogen or stabilizing the sample in Ambion's RNAlater solution. Some researchers archive tissue lysates homogenized in chaotropic lysis buffers containing, for example, guanidinium isothiocyanate, yet these lysates must also be stored at freezing temperatures to minimize RNA degradation. The invention permits the hands-free disruption of tissue to create a lysate that can be conveniently and effectively stored at room temperature when other methods are wholly unsuitable for such.

The methods and compositions of the present invention include one or a mixture of catabolic enzyme(s) or nuclease inhibitor(s), or both, that surprisingly and unexpectedly have a synergistic effect in isolating and preserving intact nucleic acid from a cell-containing sample. This can be done by degrading or liquifying the cell-containing sample in a relatively short period of time. These and other non-limiting aspects of the present invention are described in further detail in the following sections.

1. Cell-Containing Samples

A cell-containing sample can include a tissue. Tissues include a vast network of cells that are, in the case of solid tissues, sequestered from one another by an extracellular matrix. This matrix is a lattice of protein and carbohydrate that maintains tissue intactness. Extracellular matrices include several classes of macromolecules, including: 1) fibers or porous sheets of collagen; 2) elongated polysaccharides (e.g., hyaluronan); and 3) protein-polysaccharide aggregates (e.g., proteoglycans). Multiadhesive proteins that bond cells together also play an important role in the strength and rigidity of the matrix. Other proteins, such as the rubber-like elastin protein, further contribute to tissue shape and flexibility.

Once cells are separated from the matrix, access to cytosolic or nuclear components requires that biomembranes be breached. These biomembranes are composed of both phospholipids (as a bilayer) and integral membrane proteins that act as a semi-permeable barrier to salts, sugars, and other small hydrophilic molecules, and an impermeable barrier to large macromolecules. The liquefaction of tissue, in certain embodiments, requires that structural proteins, carbohydrates, and lipids be disorganized at the molecular level. This can be accomplished by chemical, enzymatic, or, to a lesser extent, mechanical methods.

In non-limiting embodiments, tissue may be a part of, or separated from, an organism. A tissue may comprise, but is not limited to, adipocytes, alveolar, ameloblasts, axon, basal cells, blood (e.g., red blood cells, white blood cells, platelets, or whole blood), blood vessel, bone, bone marrow, brain, breast, cartilage, cervix, colon, cornea, embryonic, endometrium, endothelial, epithelial, esophagus, facia, fibroblast, follicular, ganglion cells, glial cells, goblet cells, kidney, liver, lung, heart, lymph node, muscle, neuron, ovaries, pancreas, peripheral blood, prostate, skin, small intestine, spleen, stem cells, stomach, or testes.

The compositions and methods of the present invention are compatible with all types of tissues, including but not limited to, fresh, flash-frozen, fixed, RNAlater® (Ambion, Inc., Austin Tex.), or RNAlater-ICE (Ambion, Inc., Austin Tex.) preserved tissue.

2. Catabolic Enzymes

Catabolic enzymes are proteins that can break down complex structures into simple structures. In certain embodiments, a catabolic enzyme can actually destroy a particular structure or compound altogether. Any catabolic enzyme that is known to those of ordinary skill are contemplated as being useful with the present invention. Non-limiting examples include catabolic enzymes that can degrade proteins, carbohydrates, lipids, phospholipids, DNA, RNA, and other cellular and non-cellular molecules.

Proteins, for example, play formative roles as structural, adhesive, elastic, and barrier elements that are important to be destroyed in order to obtain cell dissociation and lysis. Proteases such as proteinase K is one example of a catabolic enzyme that can perform such a function. Indeed, proteinase K has been used for many years as a tool to facilitate the isolation of both RNA and DNA (Farrell 1998; Jackson 1990). As a relatively non-specific protease that retains some activity in detergents such as SDS, proteinase K can expedite the lysis of both cultured cells and intact tissue. Proteinase K-based RNA isolation methods typically use 0.1-1 mg/ml enzyme in a lysis buffer containing 2% SDS (Farrell 1998; Jackson 1990; Lai, 1993).

Lipases are responsible for the breakdown of lipids, important structural components of a cell. Lipases can attack the bond between the glycerol molecule oxygen and the fatty acid. A subset of lipases are phospholipidases which breakdown phospholipids. There are four classes of phospholipases, phospholipidase A, B, C, or D. Phospholipases usually attack a glycerol ester linkage containing any length fatty acid attached to it. The result of this digestion is a hydrophilic head molecule, glycerol and fatty acids of various chain lengths.

Collagen can be found in almost every type of tissue. Collagen proteins are used to construct collagen fibrils, and are the main components of the supporting tissue of connective tissue, bones, cartilage, teeth and extracellular matrices of skin and blood vessels. Collagenases are enzymes that are able to cleave the peptide bonds in the triple helical collagen molecule.

Elastin is a protein that provides elasticity to tissues and organs. Elastin can be found predominantly in arterial walls, lungs, intestines, and skin. It functions in a symbiotic relationship with collagen. Whereas collagen provides rigidity, elastin is the protein which allows the connective tissues in blood vessels and heart tissues, for example, to stretch and then recoil to their original positions. Elastase is an enzyme that catalyzes the hydrolysis of elastin.

About 1-10% of the cartilage glycosaminoglycans is hyaluronan. Hyaluronan can be found in other tissues such as the skin, eye, and body liquids. Hyaluronan is an unsulphated glycosaminoglycan, made of repeating disaccharide units of GlcUA and GlcNAc. Hyaluronidase is an enzyme that catalyzes the breakdown of hyaluronan in the body, thereby increasing tissue permeability to fluids.

Trypsin is an enzyme that can breakdown proteins by splitting peptide bonds on the carboxyl side of lysine and arginine residues. It is classified in the serine protease family because of the presence of a vital serine amino acid residue in the active site.

Subtilisin is an extracellular enzyme produced by certain strains of a soil bacterium (Bacillus subtilis) that catalyzes the breakdown of proteins into polypeptides and resembles trypsin in its action. There are also several different types of Subtilisin subtypes and derivatives that are described throughout this document and that are contemplated as being useful with the present invention.

Chymotrypsin is a protein-digesting enzyme that catalyzes the hydrolysis of proteins. It is selective for peptide bonds with aromatic or large hydrophobic side chains (Tyr, Trp, Phe, Met) on the carboxyl side of this bond, and it also catalyses the hydrolysis of ester bonds.

Papain is a proteolytic enzyme that is derived from papaya and certain other plants. It can breakdown proteins by cleaving the peptide bond.

DNases are capable of degrading deoxyribonucleic acid (DNA). DNases include both exonucleases and endonucleases. Non-limiting examples of DNases that can be used with the present invention include DNase I, DNase II, and shrimp arctic nuclease.

The following Table 1 provides a non-limiting summary of the different types of catabolic enzymes that can be used with the present invention.

TABLE 1

| Enzyme | Substrate | Specificity |
|---|---|---|
| Collagenase | Collagen | Pro(Hyp)-X-Gly-Pro(Hyp)- |
| Elastase | Elastin, other proteins | Ala, other neutral amino acids preferred |
| Hyaluronidase | Hyaluronan | endo-N-acetylhexosaminic bonds |
| Trypsin | General protein | Arg and Lys |
| Chymotrypsin | General protein | Tyr, Phe, and Trp |
| Papain | General protein | Arg, Lys, Phe preferred |
| Proteinase K | General protein | Broad specificity; some preference for large, |
| | | uncharged amino acids |
| Lipase | Fatty acids | Triacylglycerol |
| DNase | DNA | Broad specificity; some preference for Pyr |
| | | linkages |
| Amylase | Starch, Glycogen, and | hydrolysis of O-glycosyl bond |
| | Dextrin | |
| Cellulase | Cellulose | hydrolysis of O-glycosyl bond |
| Alcalase | General protein | Broad specificity, some preference for peptide |
| | | amines, hemoglobin, endoprotease |
| FLAVOURZYME ™ | General protein | Broad specificity, some preference for |
| | | Carboxypeptidase |
| Keratinase | General protein | Broad specificity, some preference for keratin |
| Subtilisin | General protein | Broad specificity, hydrolysis of peptide bonds |
| PEG-Subtilisin | General protein | Broad specificity, preference for a large |
| | | uncharged residue |
| Subtilisin Carlsberg | General protein | Broad specificity, preference for a large |
| | | uncharged residue |
| Properase | General protein | Broad specificity, preference for a large |
| | | uncharged residue |
| Purafect OX | General protein | Broad specificity, preference for a large |
| | | uncharged residue |
| Viscozyme | Carbohydrates | Cell walls |
| Pronase | General protein | Broad specificity |
| Blendzyme 1, 3, and 4 | General protein | Broad specificity; some preference for |
| | | collagen |
| Pepsin A | General protein | C-terminal to F, L and E |
| Recombinant | General protein | Broad specificity; some preference for large, |
| proteinase K | | uncharged amino acids |
| Type 1A collagenase | Collagen, gelatin | hydrolysis of peptide bond |
| Esperase | General protein | Broad specificity, hydrolysis of peptide bonds |
| Everlase | General protein | Broad specificity, preference for a large |
| | | uncharged residue |
| Neutrase | General protein | Broad specificity, hydrolysis of peptide bonds |
| Savinase | General protein | Broad specificity, hydrolysis of peptide bonds |
| Glucanex | cellulase, protease, and | beta-glucan polysaccharides |
| | chitinase | |
| Thermolysin | General Protein | Xaa-Leu > Xaa-Phe, Gly-Leu-NH2 |

It is contemplated that the catabolic enzymes discussed above and throughout this document can be used in conjunction with other aspects of the present invention. For example, the inventors have discovered that combinations of catabolic enzymes with nuclease inhibitors have a synergistic effect that can be used to isolate nucleic acids from a cell-containing sample.

3. Nuclease Inhibitors

Nuclease inhibitors are compounds that can inhibit or reduce the effects of nucleases. Nucleases are a class of enzymes that degrade DNA and/or RNA molecules by cleaving the phosphodiester bonds that link adjacent nucleotides. In deoxyribonuclease (DNase), for example, the substrate is DNA. By contrast, the substrate for ribonuclease (RNase) is RNA. Nucleases can further be classified as an endonuclease (i.e., cleaving internal sites in the substrate molecule) or an exonuclease (i.e. progressively cleaving from the end of the substrate molecule).

Nuclease inhibitors come in a variety of forms and substances (e.g., detergents, small molecules, proteinaceous compounds, non-proteinaceous compounds, nuclease antibodies). The following sections provide non-limiting examples of the different types of nuclease inhibitors that are contemplated as being useful with the present invention.

i. Detergents

Detergents can be used with the present invention to inhibit or reduce the activity of nucleases. Detergents exhibit a synergistic effect with other anti-nucleases to enhance the activity of the other anti-nucleases. Detergents are amphipathic molecules with an apolar end of aliphatic or aromatic nature and a polar end which may be charged or uncharged. Detergents are more hydrophilic than lipids and thus have greater water solubility than lipids. They allow for the dispersion of water insoluble compounds into aqueous media and can be used to isolate and purify proteins in a native form.

A “detergent” includes, for example, ionic (e.g., cationic, anionic, and zwitterionic) and non-ionic surfactants. Non-limiting examples cationic surfactants include DMDAO or other amine oxides, long-chain primary amines, diamines and polyamines and their salts, quaternary ammonium salts, polyoxyethylenated long-chain amines, and quaternized polyoxyethylenated long-chain amines.

Non-limiting examples of anionic surfactants include a dodecyl sulfate detergents (e.g., sodium dodecyl sulfate (SDS)), salts of carboxylic acids (i.e. soaps), salts of sulfonic acids, salts of sulfuric acid, phosphoric and polyphosphoric acid esters, alkylphosphates, monoalkyl phosphate (MAP), and salts of perfluorocarboxylic acids.

Non-limiting examples of zwitterionic surfactants include cocoamidopropyl hydroxysultaine (CAPHS) and others which are pH-sensitive and require special care in designing the appropriate pH of the formula (i.e. alkylaminopropionic acids, imidazoline carboxylates, and betaines) or those which are not pH-sensitive (i.e. sulfobetaines, sultaines).

Non-limiting examples of non-ionic surfactants include alkylphenol ethoxylates, alcohol ethoxylates, polyoxyethylenated polyoxypropylene glycols, polyoxyethylenated mercaptans, long-chain carboxylic acid esters, alkonolamides, tertiary acetylenic glycols, polyoxyethylenated silicones, N-alkylpyrrolidones, and alkylpolyglycosidases.

These and other surfactants and detergents that are discussed throughout this document and that are known to those of skill in the art are contemplated as being useful with the present invention. Additional non-limiting examples of detergents and surfactants that can be used with the present invention include those described in McCutcheon's, Detergents and Emulsifiers, North America edition (1986), published by allured Publishing Corporation; McCutcheon's, Functional Materials, North American Edition (1992), and PCT Application Nos. PCT/US97/07012, filed on Apr. 25, 1997 and PCT/US97/07013, filed on Apr. 25, 1997, the contents of which are incorporated by reference.

In other embodiments, any combination of the detergents and surfactants discussed in this document or known to a person of skill in the art is also acceptable. For example, a surfactant can include at least one anionic and one cationic surfactant, at least one cationic and one zwitterionic surfactant, or at least one anionic and non-ionic, or other combinations which are compatible.

ii. Small Molecule Nuclease Inhibitors

Small molecules nuclease inhibitors are also contemplated as being useful with the present invention. Non-limiting examples include compounds that include an aromatic structure or a polycyclic aromatic structure, or both. Additional non-limiting examples include NCI-65828, NCI 65845, benzopurpurin B, NCI-65841, NCI 79596, NCI-9617, NCI-16224, suramin, direct red 1, NCI-7815, NCI-45618, NCI-47740, prB ZBP, NCI-65568, NCI-79741, NCI-65820, NCI-65553, NCI-58047, NCI-65847, xylidene ponceau 2R, eriochrome black T, amaranth, new coccine, acid red 37, acid violet 7, NCI-45608, NCI-75661, NCI-73416, NCI-724225, orange G, NCI 47755, sunset yellow, NCI-47735, NCI-37176, violamine R, NCI-65844, direct red 13, NCI-45601, NCI 75916, NCI-65546, NCI-65855, NCI-75963, NCI-45612, NCI-8674, NCI-75778, NCI-34933, NCI-1698, NCI-7814, NCI-45550, NCI-77521, cefsulodin, NCI-174066, NCI-12455, NCI-45541, NCI-79744, NCI-42067, NCI-45571, NCI-45538, NCI-45540, NCI-9360, NCI-12857, NCI-D726712, NCI-45542, NCI-7557, S321443, NCI-224131, NCI-45557, NCI-1741, NCI-1743, NCI-227726, NCI-16163, NCI-16169, NCI-88947, NCI-17061, NCI-37169, beryllon II, CB-0181431, CB-473872, JLJ-1, JLJ-2, JLJ-3, CB-467929, CB-534510, CB-540408, CB-180582, CB-180553, CB-186847, CB-477474, CB-152591, NCI-37136, NCI-202516, CB-039263, CB-181145, CB-181429, CB-205125, and CB-224197. CB is ChemBridge Corporation and NCI is National Cancer Institute. The structures of these compounds and additional small molecule inhibitors are disclosed in U.S. application Ser. No. 10/786,875, filed on Feb. 25, 2004, entitled “Improved Nuclease Inhibitor Cocktail” by Latham et al. The contents of this application is incorporated by reference.

Derivatives of these small molecule inhibitors are also contemplated as being useful with the present invention. Chemical modifications may also be made to these compounds. Chemical modifications may be advantageous, for example, to increase or decrease the inhibitory efficacy of these compounds. A person of ordinary skill in the art would be able to recognize and identify acceptable known and unknown derivatives and/or chemical modifications that can be made to these compounds without undue experimentation.

Other non-limiting examples of nuclease inhibitors, including small-molecule nuclease inhibitors, and including derivatives and chemical modifications of the compounds noted above, that can be used with the methods, compositions, reagents, and kits of the present invention can be found: (i) throughout this specification (ii); in provisional application Ser. No. 60/547,721, filed Feb. 25, 2004, which is entitled “Nuclease Inhibitors for Use in Biological Applications” by Latham et al.; and (iii) in PCT application entitled “Small-Molecule Inhibitors of Angiogenin and In Vivo Anti-Tumor Compounds” by Shapiro et al., filed on Feb. 25, 2004, which claims the benefit of U.S. provisional application Ser. No. 60/449,912, filed Feb. 25, 2003. The entire text of these applications are incorporated by reference.

4. Equivalents

Known and unknown equivalents to the compounds discussed throughout this specification can be used with the compositions and methods of the present invention. The equivalents can be used as substitutes for the specific compounds and/or be added to the methods and compositions of the present invention. A person of ordinary skill in the art would be able to recognize and identify acceptable known and unknown equivalents to the specific compounds without undue experimentation.

5. Isolation and Purification of Nucleic Acids

The present invention can be used to obtain nucleic acids such as RNA from a tissue sample. The obtained RNA, for example, can subsequently be purified by any number of means that are known to those of skill in the art (Sambrook et al., 1989). Non-limiting purification procedures include Polyacrylamide Gel Electrophoresis, High Performance Liquid Chromatography (HPLC), Gel chromatography or Molecular Sieve Chromatography, Affinity Chromatography, cesium chloride centrifugation gradients, solid supports or resins. These and other purification techniques that can be used with the present invention are described in U.S. application Ser. No. 10/955,974, filed Sep. 30, 2004, entitled “Modified Surfaces as Solid Supports for Nucleic Acid Purification” by Latham et al., the text of which is incorporated by reference.

The term “isolated nucleic acid” includes a nucleic acid molecule (e.g., an RNA or DNA molecule) that has been isolated free of or essentially free of the bulk of the total genomic and transcribed nucleic acids, cellular components, in vitro reaction components, or small biological molecules, or the like from one or more cells or tissue samples.

6. Uses of Nucleic Acid Obtained from Cell-Containing Samples

Nucleic acids that are obtained from cell-containing samples can be used in a number of molecular biological applications known to those of skill in the art ranging from amplification, isolation, digestion, translation, or transcription reactions (Sambrook et al., 2001; Maniatis et al. 1990). Additionally, nucleic acids such as RNA obtained from tissue samples may be analyzed or quantitated by various methods that are known to those of skill in the art. These and other aspects of the present invention are described in further detail in the following sections.

i. Quantitative PCR

Two approaches, competitive quantitative PCR™ (QPCR) and real-time quantitative PCR™, both estimate target gene concentration in a sample by comparison with standard curves constructed from amplifications of serial dilutions of standard RNA. However, they differ substantially in how these standard curves are generated. In competitive QPCR, an internal competitor RNA is added at a known concentration to both serially diluted standard samples and unknown (environmental) samples. After coamplification, ratios of the internal competitor and target PCR™ products are calculated for both standard dilutions and unknown samples, and a standard curve is constructed that plots competitor-target PCR™ product ratios against the initial RNA concentration of the standard dilutions. Given equal amplification efficiency of competitor and RNA, the concentration of the latter in environmental samples can be extrapolated from this standard curve.

In real-time QPCR, the accumulation of amplification product is measured continuously in both standard dilutions of RNA and samples containing unknown amounts of RNA. A standard curve is constructed by correlating initial template concentration in the standard samples with the number of PCR™ cycles (Cₜ) necessary to produce a specific threshold concentration of product. In the test samples, the target PCR™ product accumulation is measured after the same Cₜ, which allows interpolation of target RNA concentration from the standard curve. Although real-time QPCR permits more rapid and facile measurement of RNA during routine analyses, competitive QPCR remains an important alternative for quantification in environmental samples. The coamplification of a known amount of competitor RNA with target RNA is an intuitive way to correct for sample-to-sample variation of amplification efficiency due to the presence of inhibitory substrates and large amounts of background RNA that are obviously absent from the standard dilutions.

Another type of QPCR is applied quantitatively PCR™. Often termed “relative quantitative PCR,” this method determines the relative concentrations of specific nucleic acids.

In PCR™, the number of molecules of the amplified RNA increase by a factor approaching two with every cycle of the reaction until some reagent becomes limiting. Thereafter, the rate of amplification becomes increasingly diminished until there is no increase in the amplified target between cycles. If a graph is plotted in which the cycle number is on the X axis and the log of the concentration of the amplified RNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified RNA becomes asymptotic to some fixed value. This is said to be the plateau portion of the curve.

The concentration of the RNA in the linear portion of the PCR™ amplification is directly proportional to the starting concentration of the target before the reaction began. By determining the concentration of the amplified products of the RNA in PCR™ reactions that have completed the same number of cycles and are in their linear ranges, it is possible to determine the relative concentrations of the specific target sequence in the original RNA mixture. If the RNA mixtures are cDNAs synthesized from RNAs isolated from different tissues, the relative abundance of the specific mRNA from which the target sequence was derived can be determined for the respective tissues. This direct proportionality between the concentration of the PCR™ products and the relative RNA abundance's is only true in the linear range of the PCR™ reaction.

The final concentration of the RNA in the plateau portion of the curve is determined by the availability of reagents in the reaction mix and is independent of the original concentration of target DNA. Therefore, the first condition that must be met before the relative abundance of a RNA species can be determined by RT-PCR for a collection of RNA populations is that the concentrations of the amplified PCR™ products must be sampled when the PCR™ reactions are in the linear portion of their curves.

The second condition that must be met for a quantitative RT-PCR experiment to successfully determine the relative abundance of a particular RNA species is that relative concentrations of the amplifiable cDNAs must be normalized to some independent standard. The goal of an RT-PCR experiment is to determine the abundance of a particular RNA species relative to the average abundance of all RNA species in the sample.

Most protocols for competitive PCR™ utilize internal PCR™ standards that are approximately as abundant as the target. These strategies are effective if the products of the PCR amplifications are sampled during their linear phases. If the products are sampled when the reactions are approaching the plateau phase, then the less abundant product becomes relatively over represented. Comparisons of relative abundance made for many different RNA samples, such as is the case when examining RNA samples for differential expression, become distorted in such a way as to make differences in relative abundance of RNAs appear less than they actually are. This is not a significant problem if the internal standard is much more abundant than the target. If the internal standard is more abundant than the target, then direct linear comparisons can be made between RNA samples.

The above discussion describes theoretical considerations for an RT-PCR assay for clinically derived materials. The problems inherent in clinical samples are that they are of variable quantity (making normalization problematic), and that they are of variable quality (necessitating the co-amplification of a reliable internal control, preferably of larger size than the target). Both of these problems are overcome if the RT-PCR is performed as a relative quantitative RT-PCR with an internal standard in which the internal standard is an amplifiable cDNA fragment that is larger than the target cDNA fragment and in which the abundance of the RNA encoding the internal standard is roughly 5-100 fold higher than the RNA encoding the target. This assay measures relative abundance, not absolute abundance of the respective RNA species.

Other studies may be performed using a more conventional relative quantitative RT-PCR assay with an external standard protocol. These assays sample the PCR™ products in the linear portion of their amplification curves. The number of PCR™ cycles that are optimal for sampling must be empirically determined for each target cDNA fragment. In addition, the reverse transcriptase products of each RNA population isolated from the various tissue samples must be carefully normalized for equal concentrations of amplifiable cDNAs. This consideration is very important since the assay measures absolute mRNA abundance. Absolute RNA abundance can be used as a measure of differential gene expression only in normalized samples. While empirical determination of the linear range of the amplification curve and normalization of cDNA preparations are tedious and time consuming processes, the resulting RT-PCR assays can be superior to those derived from the relative quantitative RT-PCR assay with an internal standard.

One reason for this advantage is that without the internal standard/competitor, all of the reagents can be converted into a single PCR™ product in the linear range of the amplification curve, thus increasing the sensitivity of the assay. Another reason is that with only one PCR product, display of the product on an electrophoretic gel or another display method becomes less complex, has less background and is easier to interpret.

ii. Microarrays

RNA obtained from a tissue sample may be analyzed using microarray technology. Microarrays are known in the art and general include a surface to which probes that correspond in sequence to gene products (e.g., cDNAs, mRNAs, cRNAs, polypeptides, and fragments thereof), can be specifically hybridized or bound at a known position. In one embodiment, the microarray is an array (i.e., a matrix) in which each position represents a discrete binding site for a product encoded by a gene (e.g., a protein or RNA), and in which binding sites are present for products of most or almost all of the genes in the organism's genome. In certain aspects, the “binding site” is a nucleic acid or nucleic acid analogue to which a particular cognate cDNA can specifically hybridize. The nucleic acid or analogue of the binding site can be, e.g., a synthetic oligomer, a full-length cDNA, a less-than full length cDNA, or a gene fragment.

The nucleic acid or analogue can be attached to a solid support, which may be made from glass, plastic (e.g., polypropylene, nylon), polyacrylamide, nitrocellulose, or other materials. One method for attaching the nucleic acids to a surface is by printing on glass plates, as is described generally by Schena et al., 1995. See also DeRisi et al., 1996; Shalon et al., 1996; Schena et al., 1996. Other methods for making microarrays, e.g., by masking (Maskos et al., 1992), may also be used. In principal, any type of array, for example, dot blots on a nylon hybridization membrane (Sambrook et al., 1989), can be used.

iii. Denaturing Agarose Gel Electrophoresis

RNA extracted from a tissue sample may be quantitated by agarose gel electrophoresis using a denaturing gel system. A positive control should be included on the gel so that any unusual results can be attributed to a problem with the gel or a problem with the RNA under analysis. RNA molecular weight markers, an RNA sample known to be intact, or both, can be used for this purpose. It is also a good idea to include a sample of the starting RNA that was used in the enrichment procedure.

Ambion's NorthernMax™ reagents for Northern Blotting include everything needed for denaturing agarose gel electrophoresis. These products are optimized for ease of use, safety, and low background, and they include detailed instructions for use. An alternative to using the NorthernMax™ reagents is to use a procedure described in “Current Protocols in Molecular Biology”, Section 4.9 (Ausubel et al., 1994). It is more difficult and time-consuming than the Northern-Max method, but it gives similar results.

iv. Assessing the Intactness of RNA

The determination of the quality or intactness of the RNA can be performed by several methods described in this application and by methods known to those of ordinary skill in the art. For example, the intactness of the RNA can be determined by using the Agilent 2100 Bioanalyzer software, whereby the area encompassed by the 18S and 28S rRNA peaks relative to the baseline are marked and the area within said marked region is quantified and compared. Other methods can include measuring the 28S/18S ratio of an isolated sample. This can be performed, for example, by gel analysis. In certain aspects of the present invention, “intact” RNA includes a 28S/18S ratio of about greater than or equal to about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0. In some cases, intactness will not be a long term consideration. This can occur in situations, for example, where after the treatment or digestion of the cell-containing sample, the RNA will be immediately, or soon thereafter, isolated or otherwise used for its intended purpose.

In other aspects, the RNA Integrity Number (RIN) generated by Agilent's Expert 2100 beta Software (imports files generated by the Agilent 2100 Bioanalyzer software) can be used to determine the intactness of the RNA. In addition, freely available software for assessing RNA integrity called the Degradometer (Ohio State University) provides quantitative information about the RNA integrity. The 2100 bioanalyzer chip file can be exported to the Degardometer Software to produce a Degradation Factor. If the RNA concentration is too low to be assessed by electrophoretic approaches (<200 pg/ul), then a crude measure of RNA intactness can be made by RT-PCR, whereby larger amplicons cannot be synthesized from RNA targets that are highly degraded. Northern blots can be useful for evaluating RNA intactness, since degradation of the full-length target RNA is readily apparent upon detection and subsequent analysis.

Additional analysis can be performed by determining if the RNA is useful in procedures that require intact RNA. Non-limiting examples of relevant procedures include RNA or DNA amplification reactions, RNA or DNA labeling reactions, RNA or DNA isolation reactions, RNA or DNA digestion reactions, in vitro translation reactions, in vitro transcription reactions, reverse transcription reactions, in vitro coupled transcription/translation reactions, or any nucleic acid based detection method that is based on hybridization (e.g., southern blotting, microarray detection, or ribonuclease protection assays).

v. Assessing RNA Yield by UV Absorbance

The concentration and purity of RNA can be determined by diluting an aliquot of the preparation (usually a 1:50 to 1:100 dilution) in TE (10 mM Tris-HCl pH 8, 1 mM EDTA) or water, and reading the absorbance in a spectrophotometer at 260 nm and 280 nm.

An A₂₆₀ of 1 is equivalent to 40 μg RNA/ml. The concentration (μg/ml) of RNA is therefore calculated by multiplying the A₂₆₀×dilution factor×40 μg/ml. The following is a typical example: The typical yield from 10 μg total RNA is 3-5 μg. If the sample is re-suspended in 25 μl, this means that the concentration will vary between 120 ng/μl and 200 ng/μl. One μl of the prep is diluted 1:50 into 49 μl of TE. The A₂₆₀=0.1. RNA concentration=0.1×50×40 μg/ml=200 μg/ml or 0.2 μg/μl. Since there are 24 μl of the prep remaining after using 1 μl to measure the concentration, the total amount of remaining RNA is 24 μl×0.2 μg/μl=4.8 μg.

vi. Assessing RNA Yield with RiboGreen®

Fluorescence-based assays may also be employed for quantitation of RNA. For example, the Molecular Probes' RiboGreen® fluorescence-based assay for RNA quantitation can be employed to measure RNA concentration. RiboGreen reagent exhibits >1000-fold fluorescence enhancement and high quantum yield (0.65) upon binding nucleic acids, with excitation and emission maxima near those of fluorescein. Unbound dye is essentially nonfluorescent and has a large extinction coefficient (67,000 cm−1 M−1). The RiboGreen assay allows detection of as little as 1.0 ng/ml RNA in a standard fluorometer, filter fluorometer, or fluorescence microplate reader-surpassing the sensitivity achieved with ethidium bromide by 200-fold. The linear quantitation range for RiboGreen reagent extends over three orders of magnitude in RNA concentration. However, RiboGreen also fluoresces when bound to DNA, thus accurate quantification of RNA is only possible when significant contaminating DNA is not present.

vii. Antisense RNA (aRNA) Amplification

Antisense RNA amplification involves reverse transcribing RNA samples with an oligo-dT primer that has a transcription promoter such as the T7 RNA polymerase consensus promoter sequence at its 5′ end (U.S. Pat. Nos. 5,514,545 and 5,545,522). First strand reverse transcription creates single-stranded cDNA. Following first strand cDNA synthesis, the template RNA that is hybridized to the cDNA is partially degraded creating RNA primers. The RNA primers are then extended to create double-stranded DNAs possessing transcription promoters. The population is transcribed with an appropriate RNA polymerase to create an RNA population possessing sequence from the cDNA. Because transcription results in tens to thousands of RNAs being created from each DNA template, substantive amplification can be achieved. The RNAs can be labeled during transcription and used directly for array analysis, or unlabeled aRNA can be reverse transcribed with labeled dNTPs to create a cDNA population for array hybridization. In either case, the detection and analysis of labeled targets are well known in the art.

Other methods of amplification that may be employed include, but are not limited to, polymerase chain reaction (referred to as PCR™; see U.S. Pat. Nos. 4,683,195, 4,683,202 and 4,800,159, and Innis et al., 1988); and ligase chain reaction (“LCR”), disclosed in European Application No. 320 308, U.S. Pat. Nos. 4,883,750, 5,912,148. Qbeta Replicase, described in PCT Application No. PCT/US87/00880, may also be used as an amplification method. Alternative methods for amplification of a nucleic acid such as RNA are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291, 5,916,779 and 5,942,391, GB Application No. 2 202 328, and in PCT Application No. PCT/US89/01025, PCT Application WO 89/06700, PCT Application WO 88/10315, European Application No. 329 822, Kwoh et al., 1989; Frohman, 1990; Ohara et al., 1989; and Walker et al., 1992 each of which is incorporated by reference.

viii. cDNA Library Construction

cDNA libraries may also be constructed and used to analyze RNA extracted from a tissue or cell sample. Construction of such libraries and analysis of RNA using such libraries may be found in Sambrook et al. (2001); Maniatis et al. (1990); Efstratiadis et al. (1976); Higuchi et al. (1976); Maniatis et al. (1976); Land et al. (1981); Okayama et al. (1982); Gubler et al. (1983); Ko (1990); Patanjali et al. (1991); U.S. Patent Appln. 20030104468, each incorporated by reference.

The cDNA libraries can subsequently be used, for example, in screening applications such as high throughput assays, including microarrays. A non-limiting example of such an array includes chip-based nucleic acid technologies such as those described by Hacia et al. (1996) and Shoemaker et al. (1996). Briefly, these techniques involve quantitative methods for analyzing large numbers of genes rapidly and accurately. By using fixed probe arrays, one can employ chip technology to segregate target molecules as high density arrays and screen these molecules on the basis of hybridization (see also, Pease et al., 1994; and Fodor et al, 1991). The term “array” as used herein refers to a systematic arrangement of nucleic acid. For example, a nucleic acid population that is representative of a desired source (e.g., human adult brain) is divided up into the minimum number of pools in which a desired screening procedure can be utilized to detect or deplete a target gene and which can be distributed into a single multi-well plate. Arrays may be of an aqueous suspension of a nucleic acid population obtainable from a desired mRNA source, comprising: a multi-well plate containing a plurality of individual wells, each individual well containing an aqueous suspension of a different content of a nucleic acid population. Examples of arrays, their uses, and implementation of them can be found in U.S. Pat. Nos. 6,329,209, 6,329,140, 6,324,479, 6,322,971, 6,316,193, 6,309,823, 5,412,087, 5,445,934, and 5,744,305, which are herein incorporated by reference.

7. Kits

In further embodiments of the invention, there is a provided a kit. Any of the compositions described herein may be comprised in a kit. In a non-limiting example, reagents, catabolic enzymes, and/or nuclease inhibitors for extracting RNA from a cell-containing sample, or for analyzing or quantitating the obtained nucleic acid can be included in the kit. The kits will thus comprise, in suitable container means, any of the reagents disclosed herein. It may also include one or more buffers, such as digestion buffers or a extracting buffers, and components for isolating the resultant nucleic acid.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit (they may be packaged together), the kit also will generally contain a second, third or other additional container into which the additional components may be separately placed. However, various combinations of components may be comprised in a vial. The kits of the present invention also will typically include a means for containing the components in close confinement for commercial sale. Such containers may include injection or blow-molded plastic containers into which the desired vials are retained. When the components of the kit are provided in one and/or more liquid solutions, the liquid solution is an aqueous solution, with a sterile aqueous solution being particularly preferred.

The components of the kit may be provided as dried powder(s). When reagents and/or components are provided as a dry powder, the powder can be reconstituted by the addition of a suitable solvent. It is envisioned that the solvent may also be provided in another container means. The container means will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

Such kits may also include components that facilitate isolation of the extracted nucleic acid. It may also include components that preserve or maintain the nucleic acid or that protect against its degradation. Such components include, but are not limited to, salts, buffers, detergents, nucleases (RNases and DNases), catabolic enzymes, RNA and/or DNA binding beads, chelating agents (e.g., EDTA), alcohol (e.g., ethanol or isopropanol), water and nuclease-free water, or glycerol, or other components, compounds, ingredients, and substances that are discussed throughout this document.

A kit can also include instructions for employing the kit components as well the use of any other reagent not included in the kit. Instructions may include variations that can be implemented.

### EXAMPLES

The following examples are included to demonstrate certain embodiments of the invention. It should be appreciated by those of skill in the art that the techniques disclosed in the examples which follow represent techniques discovered by the inventors to function well in the practice of the invention, and thus can be considered to constitute some modes for its practice. However, those of skill in the art should, in light of the present disclosure, appreciate that many changes can be made in the specific embodiments which are disclosed and still obtain a like or similar result without departing from the spirit and scope of the invention.

### Example 1

### Criteria for the Isolation and Analysis of RNA

The inventors have developed compositions and methods that can be used to obtain intact nucleic such as RNA from a variety of cell-containing samples. The extraction, isolation, and quantification of the nucleic acid can be performed in an efficient manner and in a relatively short period of time. A determination of the level of quality or intactness of the RNA can also be measured in an efficient and accurate manner. The following provides a non-limiting example of how to perform these steps.

Extraction: The extraction of RNA from a cell-containing sample can be performed in a number of ways such as those disclosed throughout this specification and those known to a person of ordinary skill in the art. In one example, the inventors obtained a whole liver of a mouse and dissected it into fragments of up to 10 mg. One fragment of mouse liver was added to a solution (100 ul) comprising 10 mM CHES pH 9.0, 2 mM CaCl₂, 0.1 mM EDTA, 2% SDS, 0.4 mg/ml Proteinase K, and 0.4 mg/ml Subtilisin. It should be recognized, however, that the solution can be varied by adding, removing, or substituting components based on the type and size of the tissue sample. The sample was then inserted into a microtube foam vortex adapter and incubated at room temperature (20-25° C.) with rapid shaking on a Vortex Genie-2 setting #6 to dissociate the tissue mass into a liquid state. As disclosed throughout this document, there are a variety of shaking devices that can be used to dissociate a cell-containing sample into a liquid state.

Isolation: Isolating the RNA from the lysate can be performed by any number of known techniques including, but not limited to polyacrylamide gel electrophoresis, high performance liquid chromatography (HPLC), gel chromatography or molecular sieve chromatography, affinity chromatography, cesium chloride centrifugation gradients, or the use of solid supports, beads, or resins.

The inventors, for example, in a non-limiting embodiment, isolated the RNA from the sample by incubating the lysate for about 10 minutes at room temperature (20-25° C.) and then using centrifugation>10,000×g for 3 minutes. Centrifugation procedures, of course, can vary depending on the type and/or amount of the tissue sample. The lysate was subsequently transferred to a new tube. The following reagents were then added to capture the RNA: 100 ug Nanomag®-250D beads as well as 200 ul of 1.6 M NaCl, 17 mM Tris-HCl pH 8.0, 75 mM β-mercaptoethanol, and 33% ethanol. It is contemplated, however, that these reagents can be varied by adding, removing, or substituting these components to conform with all of the different types of tissue samples that can be used with the present invention. The binding reaction occurred for a period of about 3 minutes to maximize binding. Then the tube was transferred to a magnetic stand to capture the beads, remove the supernatant, and wash the beads two times with 300 ul of 10 mM KCl, 2 mM Tris-HCl pH 7.0, 0.2 mM EDTA, and 80% ethanol. Washing solutions can be varied based on the specifics of each assay. Following the wash steps, the RNA was eluted from the magnetic beads with 20 ul of 5 mM KCl and 0.2 mM EDTA pH 8.0, preheated to 58-60° C. Elution solutions can also vary depending, for example, on the amount and/or type of tissue sample. The genomic DNA was removed with the addition of 20 Units of recombinant DNase I (Ambion, Inc., Austin, Tex.) in 100 ul of 1×DNase I buffer during an incubation period of 20 minutes at room temperature with gentle agitation. Alternatively, the genomic DNA can be removed by the addition of 10-20 Units of TURBO DNase (Ambion, Inc., Austin, Tex.) in a solution containing 1×DNase I buffer supplemented with 150-250 mM NaCl. The buffer solutions, of course, can vary. Following the DNase digestion step to remove the DNase and DNA fragments from the RNA, reagents were added to recapture the RNA onto the beads, then washed 2 more times, and the RNA was eluted in 20 ul (as described above).

Quantification and Determination of RNA Quality/Intactness: The determination of the quality or intactness of the RNA can be performed by several methods described in this application and by methods known to those of ordinary skill in the art. For example, the intactness of the RNA can be determined by using the Agilent 2100 Bioanalyzer software, whereby the area encompassed by the 18S and 28S rRNA peaks relative to the baseline are marked and the area within said marked region is quantified and compared. Other methods can include measuring the 28S/18S ratio of an isolated sample. This can be performed, for example, by gel analysis. Additional analysis can be performed by determining if the RNA is useful in procedures that requires intact RNA such as, for example, amplification reactions, isolation reactions, hybridization protection assays, or reverse transcription reactions. The RNA Integrity Number (RIN) generated by Agilent's Expert 2100 beta Software (imports files generated by the Agilent 2100 Bioanalyzer software) can be used to determine the intactness of the RNA. In addition, freely available software for assessing RNA integrity called the Degradometer (Ohio State University) provides quantitative information about the RNA integrity. The 2100 bioanalyzer chip file can be exported to the Degardometer Software to produce a Degradation Factor. If the RNA concentration is too low to be assessed by electrophoretic approaches (<200 pg/ul), then a crude measure of RNA intactness can be made by RT-PCR, whereby larger amplicons cannot be synthesized from RNA targets that are highly degraded. Last, Northern blots can be useful for evaluating RNA intactness, since degradation of the full-length target RNA is readily apparent upon detection and subsequent analysis.

The inventors, for example, determined the intactness of the RNA by first measuring the concentration and purity (260/280 nm ratio) of the RNA. This was determined by reading the absorbance of an aliquot of the preparation on the Nanodrop ND-1000A UV-Vis Spectrophotometer at 260 nm and 280 nm. An A₂₆₀ of 1 is equivalent to 40 ug RNA/ml, therefore the concentration (ug/ml) of RNA was calculated by multiplying the (A₂₆₀) (dilution factor)(40 ug/ml). The amount of total RNA recovered was >12 μg per 5 mg of mouse liver with purity>1.8.

The RNA quality was determined by analyzing 1 ul of the RNA sample on an Agilent 2100 Bioanlyzer instrument with the RNA LabChip Kit as per the manufacturer's protocol. As noted, above, however, any other methods known to those of skill in the art can be used. The total RNA isolated from mouse liver tissue produced 28S/18S ratios of ≧0.7, whereby the majority of the replicates produced intact RNA with 28S/18A ratios of 1.0. Of course, it will be recognized that such ratios will vary depending on, for example, the tissue mass, tissue handling, and/or manipulation of the tissue prior to isolation and/or type of sample to be analyzed. Additionally, 28S/18S ratios of about greater than or equal to about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 can indicate intact RNA. In some cases, intactness will not be a long term consideration. This can occur in situations, for example, where after the treatment or digestion of the cell-containing sample, the RNA will be immediately, or soon thereafter, isolated or otherwise used for its intended purpose.

### Example 2

### The Enzymatic Potency of a Combination of Proteases is Superior to a Single Protease

To demonstrate the benefit of a protease cocktail, a fluorometric kinetic assay was developed to determine the synergistic activity of proteases in combination. This assay contained 2.5 μg/ml final Bodipy TR-X labeled casein substrate (Molecular Probes) in a background of unlabeled BSA, phosphorylase, lysozyme, and casein 0.6 mg/ml final each. The protein substrate was then added to the Tris-based buffer to a reaction volume of 95 μl.

The kinetic assay was initiated with 5 μl of each respective protease or combination and data was collected on a SpectraMAX GeminiXS Fluorometer (Molecular Devices). The excitation wavelength was set at 558 nm and the emission wavelength was set at 623 nm. The reaction proceeded at 30 C, and time points were collected every 45 seconds for 20 minutes. The inventors discovered that a cocktail of Proteinase K and Subtilisin Carlsberg (0.4 mg/ml each final) was at least 10% superior in activity to other individual proteases or other combinations tested in this assay (Table 2).

TABLE 2
Protease Activity*

| # | Protease | slope (RFU's/sec) |
|---|---|---|
| 1 | Proteinase K | 0.190 |
| 2 | Subtilisin | 0.245 |
| 3 | Subtilisin-PEG | 0.109 |
| 4 | Keratinase | 0.115 |
| 5 | Papain | 0.018 |
| 6 | Properase | 0.153 |
| 7 | Purafect | 0.154 |
| 8 | Purafect OX | 0.161 |
| 9 | Savinase | 0.179 |
| 10 | Everalse | 0.139 |
| 11 | Neutrase | 0.002 |
| 12 | Esperase | 0.206 |
| 13 | Viscozyme | 0.027 |
| 14 | Proteinase K, Subtilisin | 0.291 |
| 15 | Proteinase K, Subtilsin-PEG | 0.220 |
| 16 | Proteinase K, Properase | 0.191 |
| 17 | Proteinase K, Purafect | 0.176 |
| 18 | Proteinase K, Purafect OX | 0.191 |
| 19 | Proteinase K, Neutrase | 0.124 |
| 20 | Proteinase K, Viscozyme | 0.166 |

*As measured by Fluorometric Assay described above (assessed with the same input protein mass = 40 ng/μl
Other tested proteases or protease mixtures are included in Table 3A and 3B.

TABLE 3A
Catabolic Enzymes Tested

| | |
|---|---|
| Alcalase, Bacillus Licheniformis | Blendzyme 1 - Liberase Blend |
| Trypsin, Bovine Pancreas | Blendzyme 3 - Liberase Blend |
| Chymotrypsin, Bovine Pancreas | Blendzyme 4 - Liberase Blend |
| Elastase, Porcine Pancreas | Lipase, Thermomyceslanuginosus |
| Flavourzyme ™, Aspergillus oryzae | Pepsin A, Porcine Stomach |
| Hyaluronidase, Bovine Testes | Recombinant Proteinase K, |
| | Tritirachium album |
| Papain, Carica papaya | Thermolysin, Bacillus |
| | thermoproteolyticus |
| Keratinase, Bacillus licheniformis | Collagenase, Type 1A |
| PEG-Subtilisin, Bacillus | Esperase, Bacillus sp |
| licheniformis | |
| Properase, Bacillus sp engineered | Everlase, Bacillus sp |
| Purafect, Bacillus sp engineered | Neutrase, Bacillus |
| | amyloliquefaciens |
| Purafect OX, Bacillus sp engineered | Savinase, Bacillus sp |
| Viscozyme, Aspergillus sp | Glucanex, Trichoderma harzianum |
| Pronase, Streptomyces griseus | |

TABLE 3B
Enzyme Blends Tested

| | |
|---|---|
| Trypsin, Chymoytrypsin | Proteinase K, Purafect |
| Chymotrypsin/Collagenase | Proteinase K, Purafect OX |
| Tryspin, Chymotrypsin, Collagenase | Proteinase K, Subtilisin, Papain |
| Proteinase K, Collagenase | Proteinase K, Keratinase |
| Collagenase, Hyaluronidase | Proteinase K, Keratinase, Subtilisin |
| Collagenase, Thermolysin | Proteinase K, Keratinase, |
| | Subtilisin, Papain |
| Blendzyme 1, Proteinase K | Proteinase K, Savinase |
| Blendzyme 4, Proteinase K | Proteinase K, Neutrase |
| Subtilisin, Alcalase | Proteinase K, Alcalase |
| Subtilisin, Papain | Proteinase K, Subtilisin Carlsberg |
| Subtilisin, Keratinase | Proteinase K, Papain |
| Keratinase, Papain | Proteinase K, Viscozyme |
| Proteinase K, Properase | Proteinase K, Glucanex |
| Proteinase K, Esperase | Proteinase K, Everlase |

The inventors also found the higher activity observed in the fluorometric assay correlated with more rapid tissue digestion, to a point. Additional protease activity did not necessarily expedite tissue digestion, and in certain instances, compromised RNA intactness when assessed on Agilent's 2100 Bioanalyzer with the RNA Nano LabChip® Assay. Conversely, adding less than 40 ug each of Proteinase K and Subtilisin Carlsberg resulted in a more sluggish rate of digestion as well as reduced RNA integrity.

### Example 3

### A Combination of Detergent and Proteases to Recover Extract Intact RNA

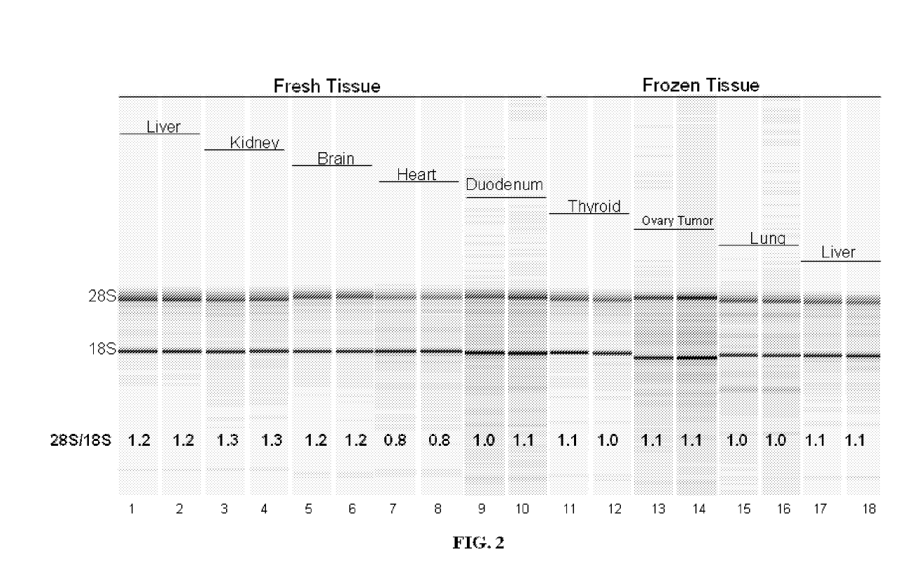

In a study to demonstrate the benefits of sodium dodecyl sulfate (SDS) in the presence of proteases to extract intact RNA from tissue, Proteinase K (0.4 mg/ml final) and Subtilisin Carlsberg (0.4 mg/ml final) were added to a Tris-based buffer containing 0.5% to 5% w/v final SDS. Inasmuch as SDS enhances apparent protease activity and inactivates RNases in solution, a condition without protease (but including SDS) was tested. The converse reaction containing proteases but no SDS was also evaluated. Up to 10 mg of frozen mouse liver tissue was added to each 100 ul reaction and incubated at room temperature with rapid shaking for 10 minutes. Following tissue digestion, the tissue lysate was purified by an RNA-binding glass-fiber filter method (RNAqueous, Ambion). The intactness of the RNA was assessed using the Agilent 2100 Bioanaylzer software after separation on an RNA LabChip®. As shown in Table 4 and FIG. 2, both proteases and SDS worked well to recover intact RNA, as indicated by the ratio of 28S to 18S ribosomal RNA peaks. A final concentration of 2% SDS produced good results in the current study. Increasing the SDS concentration further complicated the reaction by converting the entire reaction volume to foam, as well as compromising downstream RNA purification steps. SDS concentrations less than 2% did not work as well to protect RNA from highly potent RNase activity in tissues such as lung and spleen.

TABLE 4
RNA Intactness after Mouse Liver Tissue Digestion in the Presence
of SDS

| Proteases | SDS (% w/v) | 28S/18S Ratio |
|---|---|---|
| 0.4 mg/ml each | 0.5% | Not detected |
| 0.4 mg/ml each | 1% | ≦0.5 |
| 0.4 mg/ml each | 1.5% | ≦0.7 |
| 0.4 mg/ml each | 2% | ≧0.8 |
| 0.4 mg/ml each | 3% | ≦0.6 |
| 0.4 mg/ml each | 4% | Not detected |
| 0.4 mg/ml each | 5% | Not detected |
| None | 2-3% | Not detected |
| 0.4 mg/ml each | None | Not detected |
| None | None | Not detected |

### Example 4

### Ambient Reaction Temperatures Favor the Recovery of Intact RNA After Rapid Enzymatic Tissue Digestion

To demonstrate the methods of the invention during the enzymatic digestion of tissue, reaction temperatures from 4° C. to 60° C. were tested. Up to 10 mg of flash-frozen mouse liver was liquefied with Proteinase K and Subtilisin Carlsberg (0.4 mg/ml each final) in the presence of 2% SDS at 4 C, 20 C, 23 C, 25 C, 37 C, 42 C, 50 C, and 60° C. All samples were incubated with rapid shaking. The 4° C. sample was incubated in a 4° C. refrigerator. All other samples were incubated in a thermomixer (Eppendorf) to control the reaction temperature. Following tissue digestion, RNA was purified from the tissue lysates and intactness analyzed as described in Example 1. The samples incubated at 4° C. were unsuccessful at the digestion step due to SDS precipitation and reduced protease activity. As a result, very little tissue digestion occurred, even within 1 hour, and the RNA that was isolated was degraded.

Samples incubated in the ambient temperature range of 20° C. to 25° C. (i.e., approximately room temperature) digested tissue rapidly within a 10-15 minute window, and enabled the extraction of intact RNA with 28S/18S ratios≧0.8. However, tissue digestion reactions incubated at temperatures 37° C. to 60° C. (as noted in Table 5) impacted the quality of the RNA with 28S/18S ratios of ≦0.7. Digestions at 60° C. resulted significantly degraded the RNA, even though tissue digestion was completed within minutes. This method of the invention is in stark contrast with current commercial products that offer protocols for proteinase K digestion of normal or fixed tissue. In these cases, digestion is typically recommended for up to hours at a time at 50-65° C.—temperatures that do not support the recovery of intact RNA that is suitable for expression profiling applications such as microarray analyses. The combination of detergent, proteases, and shaking described in this specification, however, enables tissue digestion within minutes at ambient temperatures, and thus the recovery of high quality RNA (FIG. 2).

TABLE 5
Rapid Tissue Digestion at Ambient Temperatures and Recovery
of Intact RNA

| Reaction | |
|---|---|
| Temperature (° C.) | 28S/18S Ratio |
| 4 | Not detected |
| 20 | ≧0.8 |
| 23 | ≧0.8 |
| 25 | ≧0.8 |
| 37 | ≦0.7 |
| 42 | ≦0.7 |
| 50 | ≦0.7 |
| 60 | ≦0.3 |

### Example 5

### The Method of Tube Shaking During Tissue Digestion Affects the Rate of Liquefaction

Fragments of tissue up to 10 mg were incubated with and without shaking. Enzymatic tissue digestions incubated without shaking required an overnight incubation, consistent with current commercial protocols. Rapid shaking coupled with an appropriate mix of Proteinase K and Subtilisin Carlsberg in the presence of SDS enable up to 10 mg of tissue to be digested/disrupted within just 10 minutes. Several types of shaking apparatus were tested; these include the Thermomixer (Eppendorf), TurboMix (Scientific Industries), Mo Bio Vortex Adapter (Mo Bio Laboratories), Microtube holder vortex adapter (Troemner), and the Microtube foam rack vortex attachment (Scientific Industries). The thermomixer provides a purely orbital motion, and required >15 minutes at 1400 rpm to digest 10 mg of mouse liver. The TurboMix vortex attachment is also an orbital-like motion, but limited to 12 simultaneous samples. More than 15 minutes was required to disrupt tissue with the TurboMix. The Mo Bio Vortex Adapter is designed for horizontal tube shaking, which reduces the effectiveness of rapid shaking (horizontal position has a longer throw). Use of this adapter required larger reaction volumes and >20 minutes to liquefy tissue. The Microtube holder and the Microtube foam rack provided rapid shaking with a vertical tube orientation. Unlike the format of rigid shakers such as the thermomixer and TurboMix attachment, the flexibility of the Microtube holder and the Microtube foam rack provided a third dimension in the shaking motion that enabled rapid digestion of up to 10 mg of tissue≦10 minutes and enabled the extraction of intact RNA as measured by the Agilent 2100 Bioanalyzer. Both the microtube holder and the microtube foam rack is designed for use with the Vortex Genie-2 and 2T series, and the optimum vortex settings for tissue liquefaction are between 6 and 7 (Oust below the maximum setting). Therefore, a variety of shaking devices were effective to obtain the benefits of the present invention. However, for some embodiments, three-dimensional shaking is preferred.

### Example 6

### Identification of Reaction Conditions that Delivers the Highest Possible RNA Quality in the Least Amount of Time

Up to 10 mg of flash-frozen mouse liver was digested with Proteinase K and Subtilisin Carlsberg (0.4 mg/ml each final) in the presence of 2% SDS while varying reaction conditions. Parameters tested included pH range 3.0 to 11.0; NaCl titration 5 mM to 200 mM; CaCl₂ titration 0.2 mM to 5.2 mM; Reductants such as TCEP, DTT, BME; Detergents such as SDS, LLS, Triton X-100, NP-40, TNS/PAS, Brij 35 and 58, EDTA Titration 0.1 mM to 25 mM; Acetamide titration 1% to 5%; N,N-Dimethylacetamide titration 5% to 20%; Betaine titration 1M to 2M; Trehalose titration 300 mM to 600 mM; NDSB 195, 201, and 256 0.5 M and 1 M for each; Pluronic-68 titration 1% to 5%; and taurine titration 10 mM to 100 mM. 2 mM CaCl₂ was added to the final reaction buffer to minimize autoproteolysis. Most additives showed no significant improvement (less than 10% increase in activity) in protease activity when measured in the Fluorometric Protease Assay, except for the buffer pH increase from 8.0 (Tris-based buffer) to 9.0 (CHES-based buffer). Proteinase K and Subtilisin activity measured in a reaction buffer at pH 9.0 (in the presence of 2% SDS) showed a 20-25% increase in protease activity. The increased protease activity at pH 9.0 was further applied to enzymatic tissue digestion, which showed pH 9.0 increased protease digestion by 2-3 minutes for mouse liver (reduced overall digestion time from 10 minutes to 7-8 minutes) and reduced digestion time for mouse lung by 5-10 minutes (typically 35-45 minutes at pH 8.0). Other tissues tested include mouse brain and RNAlater treated tissue, which also digested more rapidly in the reaction buffer at pH 9.0. Thus, tissue digestion was most rapid and the recovered RNA most intact when 0.4 mg/ml Proteinase K and Subtilisin Carlsberg was included in a 10 mM CHES pH 9.0, 2 mM CalC₂, 2% SDS, 0.1 mM EDTA buffer and the tissue digestion performed with the aid of a Microtube foam vortex adaptor (FIG. 2).

### Example 7

### Identification of Dextran Magnetic Bead RNA-Binding, Wash, and Elution Conditions to Deliver the Highest Possible RNA Yield

The conditions described in Example 6 provide high yields of high quality RNA in a tissue lysate. To extract the RNA from this lysate, a procedure based on the use of Dextran Magnetic Beads was employed. This RNA-binding chemistry is described in U.S. application Ser. No. 10/955,974, filed Sep. 30, 2004, entitled “Modified Surfaces as Solid Supports for Nucleic Acid Purification” by Latham et al., the text of which is incorporated by reference. Tissue lysates were prepared enzymatically with up to 10 mg of flash-frozen mouse liver. 100 ul of the prepared lysates was then added to 100 ug of Dextran Magnetic Beads prepared in 200 ul binding solution consisting of 1.5 M NaCl, 17 mM Tris-HCl pH 8.0, 75 mM β-mercaptoethanol, 33% ethanol, which minimized bead clumping during the RNA binding step, and allowed for tight bead-pellet formation when positioned on the magnetic stand. The above mentioned formulation also ensures a clearer RNA elution with less bead contamination and less cellular contamination.

The inventors found the most common nucleic acid binding solution—one that contains chaotropic guanidinium salts—created undesirable precipitates when combined with the SDS in the tissue digestion buffer. Instead, an RNA binding solution formulated with 1.5 M NaCl, 16 mM Tris-HCl pH 8.0, and 75 mM β-Mercaptoethanol was used to denature proteins, minimize bead clumping during the RNA binding step, and ensure tight formation of the bead pellet when positioned on the magnetic stand. A wash solution comprised of 10 mM KCl, 2 mM Tris-HCl pH 7.0, and 0.2 mM EDTA, 80% ethanol helped to remove cellular contaminates. In addition, an elution solution comprised of 5 mM KCl and 0.1 mM EDTA also assisted with tight bead-pellet formation during the RNA elution process to produce the highest possible RNA yield compared to other solutions commonly used for RNA purification.

### Example 8

### Use with Multiple Tissue Types to Isolate RNA

To demonstrate the methods of the invention with many different tissue types, mouse tissues were collected fresh, flash-frozen in liquid nitrogen, or collected fresh and immediately preserved in RNAlater® (RNA stabilizing reagent). Human tissues were flash-frozen in liquid nitrogen. Up to 10 mg biopsy-sized samples of each tissue type were evaluated with the invention and analyzed as described in Example 1. As outlined in Table 6, the invention is compatible with a broad range of tissue types (FIG. 2). Non-limiting examples include soft tissue (such as liver and thyroid), fibrous tissue (heart and lung), fatty tissue (brain), and tissues soaked in RNAlater® preservative (Ambion).

TABLE 6
ELT Delivers Intact RNA Across Many Tissue Types (Ribosomal
ratios are expressed as averaged values, n ≧ 4).

| | | | | 28S/18S |
|---|---|---|---|---|
| | 28S/18S | 28S/18S | 28S/18S | Ratio |
| | Ratio | Ratio | Ratio | RNAlater- |
| Tissue Type | Fresh | Frozen | RNAlater | ICE |
| Mouse | | | | |
| Brain | ≧1.2 | ≧1.2 | ≧1.2 | NA |
| Liver | ≧1.2 | ≧1.1 | ≧1.0 | ≧1.0 |
| Kidney | ≧1.2 | ≧1.0 | ≧0.8 | NA |
| Heart | ≧0.8 | ≧0.8 | NA | NA |
| Small Intestine/Colon | ≧1.0 | ≧1.0 | NA | NA |
| Lung | ≧0.8 | ≧0.8 | NA | NA |
| Thyroid | ≧1.0 | NA | NA | NA |
| Thymus | ≧1.0 | NA | NA | NA |
| Human | | | | |
| Ovary Tumor | NA | ≧1.1 | NA | NA |
| Thyroid Tumor | NA | ≧1.1 | NA | NA |
| Brain | NA | ≧0.8 | NA | NA |

NA = not applicable

### Example 9

### Isolation of Intact RNA from Human Whole Blood

Total RNA was recovered from a sample of whole blood. A range of volumes, spanning 5% to 60% w/v final was added to ELT standard conditions, which contained a synthetic RNA tracer to monitor degradation and recovery. As described in Example 1, the samples were incubated with rapid shaking, the RNA purified and analyzed. The inventors found that intact RNA can be extracted from up to 10% w/v final whole blood.

Blood fractions such as plasma, serum, or cellular populations such as leukocytes may also be used with the invention. For example, plasma fractionated from a whole blood sample may be used with the invention to isolate intact viral or total RNA.

### Example 10

### RNA Intactness can be Preserved for at Least 6 Days at Ambient Temperatures

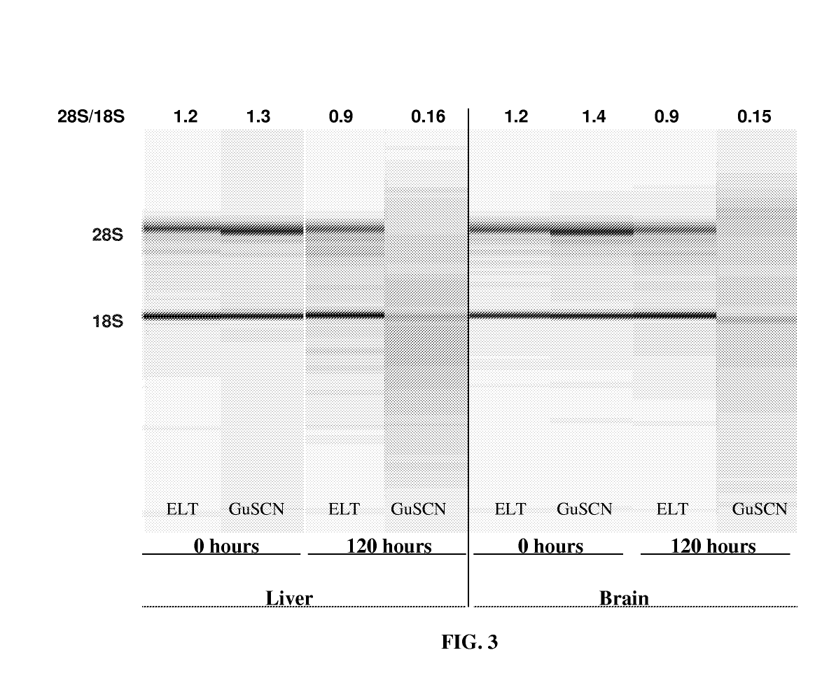

Approximately 5 mg of fresh mouse brain, fresh and flash-frozen mouse liver tissue was added to the Proteinase K and Subtilisin proteases to generate tissue lysates. The liquefied tissues was then incubated at 22-25 C (ambient temperature) over a period of 6 days and compared to tissue lysates generated with fresh mouse brain and liver disrupted in a guanadinium-based lysis solution from Ambion's RNAqueous kit. Immediately after the tissue lysates were created, a fraction of the lysate was removed and RNA purified and analyzed as described in Example 1. Subsequent time points were taken over a 6-day incubation period with additional fractions of the room temperature tissue lysate removed, purified and analyzed. It was discovered that the invention maintained the RNA integrity for nearly a week, as shown in Table 7. A comparison of the 28S/18S ratios for the frozen liver sample between day 0 (28S/18S=0.9) and day 6 (28S/18S=0.8) revealed a minimal change in RNA intactness. The RNA quality was further confirmed with analysis of 5 genes (β-actin, caspase3, p53, cdk9, and myc) in qRT-PCR with less than 0.5 Cₜ deviation (duplicates) between samples processed immediately, or stored for 6 days at room temperature. RNA isolated from guanidinium tissue lysates was partially degraded within 2 days (28S/18S=0.9) and was significantly degraded after 5 days incubation (28S/18S=0.2) at room temperature as shown in Table 7. Unlike other RNA isolation systems, the invention is capable of preserving RNA in tissue lysates at ambient temperatures for several days longer than the current solutions used to inhibit RNase during tissue dissociation (FIG. 3). FIG. 3 provides data that shows that RNA can be preserved in tissue lysates for days at room temperature when practicing the methods of the present invention. FIG. 3 also shows that the RNA in guanidine-based tissue lysates becomes degraded when preserved under the same conditions.

TABLE 7
Enzymatic Tissue Lysis Preserves Intact RNA for up to 6 days in
Tissue Lysates

| | 28S/18S Ratio | | | | |
|---|---|---|---|---|---|
| Tissue Type | Day 0 | Day 2 | Day 3 | Day 5 | Day 6 |
| Enzymatic Lysis | | | | | |
| Fresh Brain | 1.2 | 1.0 | ND | 0.9 | ND |
| Fresh Liver | 1.1 | 1.1 | ND | 0.9 | ND |
| Frozen Liver | 0.9 | ND | 0.8 | ND | 0.8 |
| Guanidinium Lysis | | | | | |
| Fresh Brain | 1.4 | 0.5 | ND | 0.2 | ND |
| Fresh Liver | 1.4 | 0.9 | ND | 0.1 | ND |

ND = Not Determined

### Example 11

### Intact RNA Isolated with the Invention is Suitable for QRT-PCR

Up to 10 mg of fresh and frozen mouse tissues (brain, liver, kidney, heart and small intestine) were processed with the invention as described in Example 1. Following isolation, 2 ng of total RNA was analyzed in real-time one-step qRT-PCR (a MMLV-RT/Taq Polymerase one tube, one buffer system). The 10 ul reactions were performed on an ABI 7900 HT Sequence Detection with standard cycling conditions. Quantification of 6 mRNA targets using TaqMan® Gene Expression Assays (ABI) (β-actin, caspase3, myc, jun, cdk9, p53) revealed less than 1 Cₜ deviation (averaged triplicates) between RNA prepared from fresh or frozen tissue.

In addition, RNA isolated with the invention was compared to two other available RNA isolation methods (RNeasy® and TRI Reagent®) and analyzed in real-time one-step qRT-PCR with the aforementioned TaqMan® Gene Expression Assays. As shown in Table 8, all targets were detected with less than 1 Cₜ deviation (averaged triplicates) among the three methods. Thus, the invention enables the isolation of RNA populations that quantified by RT-PCR to yield comparable gene expression levels with popular commercial methods.

TABLE 8
qRT-PCR Analysis of Total RNA Isolated after Enzymatic Tissue
Lysis Compared with Popular Commercial Methods*

| | Triplicate | Triplicate |
|---|---|---|
| Sample | Ct Average | Ct StDev |
| TRI Liver Fresh-1 B-actin | 21.539 | 0.159 |
| RNeasy Liver Fresh-4 B-actin | 21.737 | 0.038 |
| ELT Liver Fresh-1 B-actin | 21.533 | 0.04 |
| TRI Liver Frozen-4 Beta-actin | 21.418 | 0.07 |
| RNeasy Liver Frozen-2 Beta-actin | 21.355 | 0.095 |
| ELT Liver Frozen-4 Beta-actin | 22.578 | 0.099 |
| TRI Liver Fresh-1 p53 | 27.751 | 0.025 |
| RNeasy Liver Fresh-4 p53 | 27.994 | 0.136 |
| ELT Liver Fresh-1 p53 | 27.468 | 0.089 |
| TRI Liver Frozen-4 p53 | 27.882 | 0.396 |
| RNeasy Liver Frozen-2 p53 | 27.742 | 0.122 |
| ELT Liver Frozen-4 p53 | 28.734 | 0.225 |
| TRI Liver Fresh-1 JUN | 30.901 | 0.031 |
| RNeasy Liver Fresh-4 JUN | 30.513 | 0.136 |
| ELT Liver Fresh-1 JUN | 29.845 | 0.263 |
| TRI Liver Frozen-4 JUN | 29.834 | 0.293 |
| RNeasy Liver Frozen-2 JUN | 29.626 | 0.119 |
| ELT Liver Frozen-JUN | 29.523 | 0.273 |
| TRI Liver Fresh-1 cdk9 | 30.205 | 0.031 |
| RNeasy Liver Fresh-4 cdk9 | 30.655 | 0.103 |
| ELT Liver Fresh-1 cdk9 | 29.93 | 0.188 |
| TRI liver Frozen-4 cdk9 | 31.594 | 0.506 |
| RNeasy Liver Frozen-2 cdk9 | 31.151 | 0.161 |
| ELT liver Frozen-4 cdk9 | 30.991 | 0.28 |
| TRI Liver Fresh-1 Casp-3 | 28.815 | 0.238 |
| RNeasy Liver Fresh-4 Casp-3 | 29.252 | 0.117 |
| ELT Liver Fresh-1 Casp-3 | 28.888 | 0.076 |
| TRI Liver Frozen-4 Casp-3 | 28.838 | 0.136 |
| RNeasy Liver Frozen-2 Casp-3 | 29.207 | 0.889 |
| ELT Liver Frozen-4 Casp-3 | 29.553 | 0.207 |
| TRI Liver Fresh-1 MYC | 27.678 | 0.055 |
| RNeasy Liver Fresh-4 MYC | 28.193 | 0.061 |
| ELT Liver Fresh-1 MYC | 27.696 | 0.048 |
| TRI Liver Frozen-4 MYC | 27.588 | 0.311 |
| RNeasy Liver Frozen-2 MYC | 27.635 | 0.177 |
| ELT Liver Frozen-4 MYC | 28.061 | 0.148 |
| TRI Kidney Fresh-2 B-actin | 20.325 | 0.061 |
| RNeasy Kidney Fresh-2 B-actin | 20.961 | 0.054 |
| ELT Kidney Fresh-3 B-actin | 20.882 | 0.015 |
| TRI Kidney Frozen-1 Beta-actin | 20.472 | 0.054 |
| RNeasy Kidney Frozn-2 Beta-actin | 20.51 | 0.109 |
| ELT Kidney Frozen-1 Beta-actin | 21.411 | 0.096 |
| TRI Kidney Fresh-2 p53 | 26.542 | 0.133 |
| RNeasy Kidney Fresh-2 p53 | 27.12 | 0.042 |
| ELT Kidney Fresh-3 p53 | 26.868 | 0.06 |
| TRI Kidney Frozen-1 p53 | 26.942 | 0.448 |
| RNeasy Kidney Frozen-2 p53 | 26.871 | 0.174 |
| ELT Kidney Frozen-1 p53 | 27.406 | 0.061 |
| TRI Kidney Fresh-2 JUN | 29.74 | 0.057 |
| RNeasy Kidney Fresh-2 JUN | 29.256 | 0.152 |
| ELT Kidney Fresh-3 JUN | 29.251 | 0.112 |
| TRI kidney Frozen-1 JUN | 28.906 | 0.943 |
| RNeasy Kidney Frozen-2 JUN | 27.377 | 0.032 |
| ELT kidney Frozen-1 JUN | 28.792 | 0.221 |
| TRI Kidney Fresh-2 cdk9 | 29.529 | 0.1 |
| RNeasy Kidney Fresh-2 cdk9 | 29.737 | 0.151 |
| ELT Kidney Fresh-3 cdk9 | 29.853 | 0.095 |
| TRI Kidney Frozen-1 cdk9 | 30.282 | 0.135 |
| RNeasy Kidney Frozen-2 cdk9 | 29.82 | 0.079 |
| ELT Kidney Frozen-1 cdk9 | 30.324 | 0.171 |
| TRI Kidney Fresh-2 Casp-3 | 28.859 | 0.117 |
| RNeasy Kidney Fresh-2 Casp-3 | 29.388 | 0.085 |
| ELT Kidney Fresh-3 Casp-3 | 29.186 | 0.145 |
| TRI Kidney Frozen-1 Casp-3 | 28.491 | 0.582 |
| RNeasy Kidney Frozen-2 Casp-3 | 28.311 | 0.304 |
| ELT Kidney Frozen-1 Casp-3 | 28.534 | 0.052 |
| TRI Kidney Fresh-2 MYC | 26.979 | 0.148 |
| RNeasy Kidney Fresh-2 MYC | 27.426 | 0.114 |
| ELT Kidney Fresh-3 MYC | 27.232 | 0.161 |
| TRI Kidney Frozen-1 MYC | 26.48 | 0.298 |
| RNeasy Kidney Frozen-2 MYC | 26.694 | 0.05 |
| ELT Kidney Frozen-1 MYC | 27.458 | 0.164 |
| TRI Brain Fresh-2 beta-actin | 20.990 | 0.142 |
| RNeasy Brain Fresh-4 beta-actin | 20.437 | 0.180 |
| ELT Brain Fresh-2 beta-actin | 21.330 | 0.237 |
| TRI Brain Frozen-3/4 Beta-actin | 20.209 | 0.101 |
| TRI Brain Frozen-3/4 Beta-actin-RT | 32.463 | 0.654 |
| RNeasy Brain Frozen-3 Beta-actin | 20.816 | 0.132 |
| RNeasy Brain Frozen-3 Beta-actin-RT | Undetermined | |
| ELT Brain Frozen-2 Beta-actin | 20.595 | 0.201 |
| ELT Brain Frozen-2 Beta-actin-RT | 36.208 | 0.967 |
| TRI Brain Fresh-2 p53 | 29.247 | 0.351 |
| RNeasy Brain Fresh-4 p53 | 28.883 | 0.878 |
| ELT Brain Fresh-2 p53 | 29.090 | 0.221 |
| TRI Brain Frozen-3/4 p53* | 27.38 | 0.313 |
| RNeasy Brain Frozen-3 p53 | 28.152 | 0.146 |
| ELT Brain Frozen-2 p53 | 27.447 | 0.211 |
| TRI Brain Fresh-2 JUN | 29.040 | 0.057 |
| RNeasy Brain Fresh-4 JUN | 27.650 | 0.787 |
| ELT Brain Fresh-2 JUN | 29.403 | 0.757 |
| TRI Brain Frozen-3/4 JUN* | 29.283 | 0.667 |
| RNeasy Brain Frozen-3 JUN | 29.338 | 0.359 |
| ELT Brain Frozen-2 JUN | 29.155 | 0.56 |
| TRI Brain Fresh-2 cdk9* | 30.360 | 1.556 |
| RNeasy Brain Fresh-4 cdk9 | 29.933 | 0.327 |
| ELT Brain Fresh-2 cdk9* | 29.735 | 2.058 |
| TRI Brain Frozen-3/4 cdk9* | 30.39 | 1.067 |
| RNeasy Brain Frozen-3 cdk9 | 29.516 | 0.125 |
| ELT Brain Frozen-2 cdk9* | 28.143 | 0.372 |
| TRI Brain Fresh-2 Casp-3 | 30.610 | 0.161 |
| RNeasy Brain Fresh-4 Casp-3 | 30.043 | 0.378 |
| ELT Brain Fresh-2 Casp-3 | 30.283 | 0.348 |
| TRI Brain Frozen-3/4 Casp-3 | 30.322 | 0.184 |
| RNeasy Brain Frozen-3 Casp-3 | 30.44 | 0.226 |
| ELT Brain Frozen-2 Casp-3 | 30.502 | 0.398 |
| TRI Brain Fresh-2 MYC | 29.170 | 0.173 |
| RNeasy Brain Fresh-4 MYC | 29.083 | 0.046 |
| ELT Brain Fresh-2 MYC | 29.367 | 0.063 |
| TRI Brain Frozen-3/4 MYC | 27.863 | 0.122 |
| RNeasy Brain Frozen-3 MYC | 29.575 | 0.178 |
| ELT Brain Frozen-2 MYC | 28.006 | 0.583 |

*ELT = Enzymatic Lysis of Tissue.
TRI = TRI Reagent ®, a product of Molecular Research Center, Inc. (Cincinnati, OH).
RNeasy ® is a product of Qiagen, Inc. (Hilden, Germany).

### Example 12

### Intact RNA Isolated with the Invention is Suitable for RNA Amplification and Microarrays

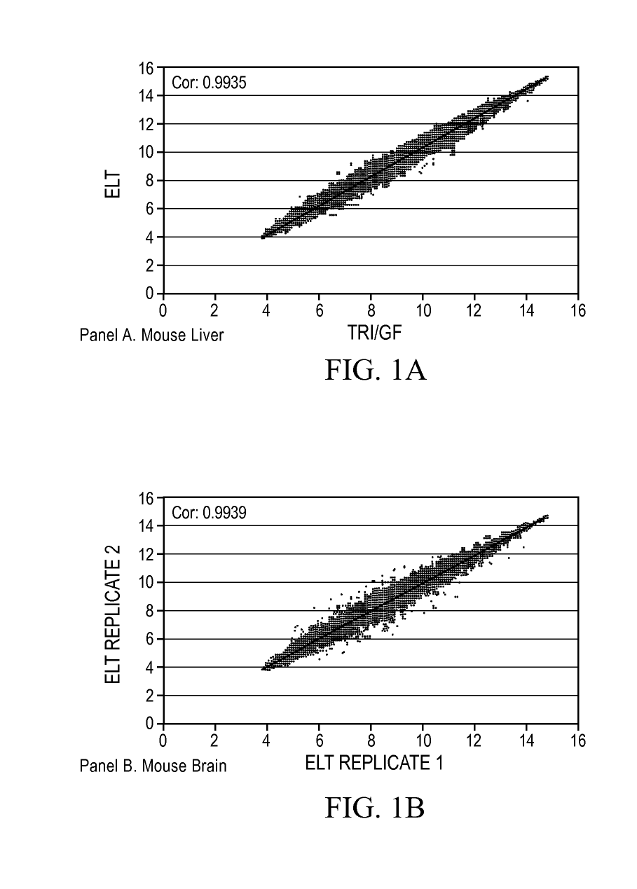

Up to 10 mg of fresh mouse liver and kidney tissues, as well as fresh and frozen mouse brain tissues, were processed as described in Example 1. Following isolation, 1 ug of total RNA from biological replicates was amplified and labeled using the MessageAmp II RNA Amplification kit (Ambion). Fragmented amplified RNA was then hybridized to Mouse Genome 430A 2.0 Arrays (Affymetrix), and scanned with a GeneChip® Scanner 3000. Data were captured and analyzed on GeneChip Operating Software (Affymetrix). Table 9 shows a comparison of RNA amplified from RNA isolated either with the invention or by the Affymetrix-recommended RNA isolation procedure (TRI® Reagent followed by glass-filter purification). The two methods of sample preparation yielded comparable microarray results that were highly correlated by several key statistical measures, such as percent present calls, total concordance (Table 10), GAPDH and β-actin 3′/5′ ratios and correlation of the normalized array signals (FIGS. 1A and 1B). In addition, biological replicates of the method of the invention were extremely well correlated. As a result, the invention enables the extraction of highly intact and highly representative RNA populations that provide comparable results with current popular methods.

TABLE 9
Comparison of RNA Amplified after Isolation from Enzyme Digested Tissue with the Affymetrix-
Recommended Eukaryotic Sample Preparation Method For Tissue

| | | | | | Fold | | |
|---|---|---|---|---|---|---|---|
| | | | | | Amplification | aRNA Size @ | |
| | | Input Total RNA | | | (Assume 3% | ~50% of Total | Array Percent |
| # | Sample Type | 28S/18S Ratio | RIN | aRNA Yield (ug) | mRNA) | Area | Present Calls |
| 1 | TRI-mLiver Fresh-1A | 1.5 | 9.2 | 99.5 | 3318 | 1730 | 57.2 |
| 2 | TRI-mLiver Fresh-1B | 1.5 | 9.2 | 79.0 | 2635 | 1935 | na |
| 3 | TRI-mLiver Fresh-3A | 1.4 | 9.4 | 74.5 | 2484 | 1870 | na |
| 4 | TRI-mLiver Fresh-3B | 1.4 | 9.4 | 82.4 | 2747 | 1855 | 56.9 |
| 5 | ELT-mLiver Fresh-1A | 1.5 | 9.2 | 78.6 | 2621 | 1560 | 58.9 |
| 6 | ELT-mLiver Fresh-1B | 1.5 | 9.2 | 64.8 | 2159 | 1450 | na |
| 7 | ELT-mLiver Fresh-2A | 1.5 | 9.3 | 72.9 | 2430 | 1565 | na |
| 8 | ELT-mLiver Fresh-2B | 1.5 | 9.3 | 73.6 | 2453 | 1560 | 58.5 |
| 9 | TRI-mKidney Fresh-1A | 1.4 | 9.2 | 111.7 | 3722 | 1855 | na |
| 10 | TRI-mKidney Fresh-1B | 1.4 | 9.2 | 127.0 | 4234 | 1800 | 63.6 |
| 11 | TRI-mKidney Fresh-2A | 1.4 | 9.1 | 119.3 | 3978 | 1800 | na |
| 12 | TRI-mKidney Fresh-2B | 1.4 | 9.1 | 139.6 | 4652 | 1855 | 62.8 |
| 13 | ELT-mKidney Fresh-3A | 1.2 | 8.3 | 77.3 | 2578 | 1450 | 64.0 |
| 14 | ELT-mKidney Fresh-3B | 1.2 | 8.3 | 75.5 | 2515 | 1510 | na |
| 15 | ELT-mKidney Fresh-4A | 1.2 | 8.5 | 77.2 | 2574 | 1500 | 65.1 |
| 16 | ELT-mKidney Fresh-4B | 1.2 | 8.5 | 78.4 | 2612 | 1485 | 66.4 |
| 17 | TRI-mBrain Fresh-2A | 1.2 | 8.9 | 106.2 | 3539 | 1780 | 63.4 |
| 18 | TRI-mBrain Fresh-2B | 1.2 | 8.9 | 104.5 | 3482 | 1865 | na |
| 19 | TRI-mBrain Fresh-3A | 1.2 | 8.7 | 93.5 | 3117 | 1805 | 63.6 |
| 20 | TRI-mBrain Fresh-3B | 1.2 | 8.7 | 111.8 | 3727 | 1855 | na |
| 21 | ELT-mBrain Fresh-3A | 1.2 | 8.9 | 127.3 | 4244 | 1920 | 64.9 |
| 22 | ELT-mBrain Fresh-3B | 1.2 | 8.9 | 114.4 | 3813 | 1920 | na |
| 23 | ELT-mBrain Fresh-4A | 1.2 | 8.8 | 120.2 | 4005 | 2040 | 64.3 |
| 24 | ELT-mBrain Fresh-4B | 1.2 | 8.8 | 85.0 | 2833 | 1575 | na |
| 25 | TRI-mBrain Frozen-1&2A | 1.3 | 8.9 | 82.9 | 2764 | 1820 | na |
| 26 | TRI-mBrain Frozen-1&2B | 1.3 | 8.9 | 85.8 | 2861 | 1855 | 63.2 |
| 27 | TRI-mBrain Frozen-3&4A | 1.3 | 9.1 | 79.8 | 2659 | 1870 | na |
| 28 | TRI-mBrain Frozen-3&4B | 1.3 | 9.1 | 84.1 | 2805 | 1855 | 62.7 |
| 29 | ELT-mBrain Frozen-2A | 1.2 | 8.7 | 120.2 | 4007 | 1925 | 64.0 |
| 30 | ELT-mBrain Frozen-2B | 1.2 | 8.7 | 115.8 | 3859 | 1920 | na |
| 31 | ELT-mBrain Frozen-3A | 1.4 | 9.1 | 105.6 | 3519 | 1930 | 65.3 |
| 32 | ELT-mBrain Frozen-3B | 1.4 | 9.1 | 108.2 | 3605 | 1870 | na |

TABLE 10
Percent total concordance and correlation after normalized microarray signal intensities*

| | Biological Replicate | Mouse | % Total | Pearsons | | |
|---|---|---|---|---|---|---|
| | Sample Name | Tissue Type | Concordance | (Correlation (r)) | r² | Avg |
| 1 | ELT 1A vs TRI 1A | Fresh Liver | 94.5 | 0.992 | 0.984 | 0.986 |
| 4 | ELT 1A vs TRI 3B | Fresh Liver | 94.5 | 0.992 | 0.984 | |
| 2 | ELT 2B vs TRI 1A | Fresh Liver | 94.5 | 0.994 | 0.987 | |
| 3 | ELT 2B vs TRI 3B | Fresh Liver | 94.8 | 0.994 | 0.988 | |
| 5 | ELT 1A vs ELT 2B | Fresh Liver | 95.4 | 0.997 | 0.994 | |
| 6 | TRI 1A vs TRI 3B | Fresh Liver | 95.6 | 0.997 | 0.995 | |
| 7 | ELT 3A vs TRI 1B | Fresh Kidney | 95.3 | 0.992 | 0.985 | 0.973 |
| 8 | ELT 3A vs TRI 2B | Fresh Kidney | 95.1 | 0.991 | 0.983 | |
| 9 | ELT 4B vs TRI 1B | Fresh Kidney | 93.6 | 0.981 | 0.962 | |
| 10 | ELT 4B vs TRI 2B | Fresh Kidney | 93.3 | 0.981 | 0.962 | |
| 11 | ELT 3A vs ELT 4B | Fresh Kidney | 94.9 | 0.992 | 0.984 | |
| 12 | TRI 1B vs TRI 2B | Fresh Kidney | 96.5 | 0.998 | 0.996 | |
| 13 | ELT 3A vs TRI 2A | Fresh Brain | 93.2 | 0.978 | 0.956 | 0.965 |
| 14 | ELT 3A vs TRI 3B | Fresh Brain | 93.5 | 0.980 | 0.961 | |
| 15 | ELT 4A vs TRI 2A | Fresh Brain | 93.7 | 0.985 | 0.970 | |
| 16 | ELT 4A vs TRI 3B | Fresh Brain | 94.1 | 0.987 | 0.973 | |
| 17 | ELT 3A vs ELT 4A | Fresh Brain | 94.8 | 0.994 | 0.988 | |
| 18 | TRI 2A vs TRI 3B | Fresh Brain | 95.7 | 0.998 | 0.996 | |
| 19 | ELT 2A vs TRI 1B | Frozen Brain | 94.2 | 0.988 | 0.976 | 0.968 |
| 20 | ELT 2A vs TRI 3B | Frozen Brain | 92.9 | 0.975 | 0.950 | |
| 21 | ELT 3A vs TRI 1B | Frozen Brain | 93.9 | 0.986 | 0.972 | |
| 22 | ELT 3A vs TRI 3B | Frozen Brain | 93.8 | 0.987 | 0.974 | |
| 23 | ELT 2A vs ELT 3A | Frozen Brain | 94.4 | 0.988 | 0.976 | |
| 24 | TRI 1B vs TRI 3B | Frozen Brain | 94.0 | 0.984 | 0.967 | |

% Total Concordance = (PP + AA)/(PP + PA + AA) normalized signal intensities
Correlation = square root of r² generated from normalized signal intensity scatterplot

### Example 13

### RNA Detection by a Hybridization-Based Assay

To determine if the invention could be used to present RNA for detection by a hybridization-based method, enzyme digested tissue was interrogated by a hybridization protection assay (HPA, manufactured by Genprobe, Inc.). In this assay, acridinium ester-containing DNA probes hybridize to the target RNA, and unhybridized single-stranded probes are degraded by the addition of a proprietary chemical solution. The fraction of targets complexed with the dye-coupled probe can then be quantified after measuring light output from the duplexed acridinium reporter in a luminometer.

To demonstrate the utility of the invention with this method, the following experiment was performed. A total of 5 mg of freshly procured mouse brain tissue was digested in triplicate as described in Example 1. Of the 300 ul total lysate volume, 100 ul was used to isolate total RNA using the mirVana PARIS kit (Ambion), and 1 ug of this RNA was assayed for the presence of microRNA's miR-124 and miR-16 by HPA using the supplier's instructions. Separately, 10-20 ul of the enzyme digested tissue lysate was assayed directly without RNA isolation. As shown in Table 11, an equivalent or greater signal intensity compared to the purified RNA was observed in the tissue lysate for both micro RNA targets. Thus, RNA targets from enzyme digested tissue lysates are receptive to both hybridization and detection, either in crude lysates or after nucleic acid purification.

TABLE 11*

| | miR-124 | | miR-16 |
|---|---|---|---|
| no RNA, H2O | 153 | no RNA, H2O | 161 |
| no RNA, tissue | 122 | 1 ug RNA purified from EDT | 450 |
| digestion buffer | | | |
| 1 ug RNA | 2898 | 1 ug RNA purified from EDT | 710 |
| purified from | | | |
| EDT | | | |
| 1 ug RNA | 2913 | 20 ul EDT | 710 |
| purified from | | | |
| EDT | | | |
| 20 ul EDT | 4219 | | |
| 20 ul EDT | 3960 | | |
| 10 ul EDT | 2401 | | |
| 10 ul EDT | 2344 | | |

*EDT = Enzyme Digested Tissue

### Example 14

### Isolation of Ribosomal, Poly(A), and Micro RNA and DNA

The method for RNA isolation is described in Example 1. Increasing the final ethanol concentration during the binding procedure to >50% will capture ribosomal, messenger, and micro RNA and genomic DNA. Poly(A) RNA selection can also be achieved by purifying RNA as described in Example 6 and enriching the mRNA by extraction from the ribosomal pool using a method such as oligo d(T) selection that is well known to one skilled in the art. In addition to RNA isolation, the methods of the invention can also be used to isolate DNA by preparing a tissue lysate as described in Example 5, degrading the RNA in the lysate with RNase A, then proceeding with a suitable DNA purification method.

### Example 15

### Long-Term Storage and Stability of a Ready-to-Use Protease Cocktail

To evaluate the use of a single tube reagent that contains all enzymatic activities, a study was performed to assess the long-term storage and stability of a ready-to-use protease cocktail comprised of Proteinase K and Subtilisin Carlsberg at 10 mg/ml each. To prepare the protease cocktail stock, equal volumes of Proteinase K (20 mg/ml) and Subtilisin Carlsberg (20 mg/ml) were combined to make a final protease cocktail concentration of 20 mg/ml. The cocktail was stored at −20 C in a storage buffer consisting of 50% glycerol, 50 mM Tris-HCl pH 8.0, and 3 mM CaCl₂. An aliquot of the protease cocktail was removed at specific intervals over a period of 16 weeks and tested for protease activity in a Fluorometric Protease Assay as described in Example 2. In Table 12, duplicate samples are averaged and the activity expressed in RFU's/sec, showing less than 15% deviation in activity between the starting time point and 16 week time point. These data show that the requisite protease activities of the invention can be conveniently stored for at least four months in a single tube.

TABLE 12
Ready-to-Use Protease Cocktail Stability. A combination of Proteinase K and Subtilisin Carlsberg
are stable and retain protease activity after storage together at −20 C for 16 weeks.*

| | | t = 0 | | t = 2 wks | | t = 6 wks | | t = 10 wks | | t = 16 wks | |
|---|---|---|---|---|---|---|---|---|---|---|---|
| | | Avg | | Avg | | Avg | | Avg | | Avg | |
| | | RFU's/ | | RFU's/ | | RFU's/ | | RFU's/ | | RFU's/ | |
| # | Condition | Sec | Stdev | Sec | Stdev | Sec | Stdev | Sec | Stdev | Sec | Stdev |
| 1 | Proteinase K | 0.206 | 0.0226 | 0.205 | 0.0410 | 0.229 | 0.0014 | 0.242 | 0.0071 | 0.200 | 0.0269 |
| 2 | Subtilisin Carlsberg | 0.247 | 0.0028 | 0.260 | 0.0226 | 0.226 | 0.0127 | 0.238 | 0.0021 | 0.220 | 0.0580 |
| 3 | Proteinase K/Subtilisin Cocktail | 0.273 | 0.0255 | 0.209 | 0.0474 | 0.246 | 0.0000 | 0.251 | 0.0127 | 0.233 | 0.0148 |
| 4 | Prot.K/Subt-Fresh Dil'n on | na | na | na | na | 0.242 | 0.0014 | 0.241 | 0.0049 | 0.258 | 0.0028 |
| | Day of Assay | | | | | | | | | | |
| 5 | Storage Buffer | 0.003 | 0.0028 | 0.004 | 0.0021 | 0.002 | 0.0000 | 0.000 | 0.0000 | 0.003 | 0.0014 |

*The results observed in the fluorometric assay mirrored the results using tissue as a substrate. The potency of tissue digestion was not compromised by 4 months of storage of the protease activities in the same tube since both the RNA yield and quality was retained (e.g., 28S/18S ratio ≧1.0 when assessed on Agilent's 2100 Bioanalyzer with the RNA Nano LabChip ® Assay).

### Example 16

### Use of Invention with Cultured Cells

Methods of the invention can be used to isolate RNA from tissue-cultured cells. For example, the invention can be used to lyse as many as 4 million HeLa cells using the conditions described in Example 5. The RNA can then be isolated and analyzed as per the methods described in Example 1. In a study performed using this method, extremely high quality total RNA was recovered, with an average 28/18S=1.6.

### Example 17

### Use of the Invention with Bacterial Cells

Methods of the invention can be used to isolate RNA from bacterial cells. For example, the invention can be used to lyse E. Coli bacteria using the conditions described in Example 5. The released RNA can then be isolated and analyzed as per the methods described in Example 1. In a study performed using this method, intact total RNA was recovered (average 23/16S=1.3) from ˜1e9 cells. Although bacterial cells possess a different cellular architecture than mammalian cells, this result revealed that the combination of detergent and potent proteases can release and protect bacterial RNA for downstream purification. In addition, lysozyme or other cell-wall digesting or permeating proteins or chemicals may be combined with the proteases or other catabolic enzymes to enable more complete RNA release and/or preservation.

### Example 18

### Enzymatic Tissue Digestion Using Non-Denaturing Solution Conditions

A disadvantage of ionic detergents and chaotropes during the processing of biological samples for RNA is that these reagents obviate direct detection of nucleic acids. Since the enzymes that amplify RNA and DNA such as reverse transcriptase and DNA polymerases are readily inactivated by such chemicals, these inhibitors must be removed prior to nucleic acid manipulation. The inventors reasoned that a non-denaturing platform for the enzymatic digestion of tissue would be particularly valuable.

The following experiment was performed. Approximately 5 mg of mouse brain, liver, or kidney tissue was digested in a buffer containing 10 mM Tris-HCl pH 8.0, 2 mM CaCl₂, 1.5 mM MgCl₂, 0.5 mM EDTA, and a protease/collagenase cocktail known as Blendzyme-4 (0.45 ug/ul final, Roche). A subset of reactions also contained 5 mM Benzopurpurin B (BpB), a recently described small molecule inhibitor of RNase A (Shapiro et al). Samples were incubated in a Thermomixer for 10-15 min at 37 C, and the RNA isolated using the RNAqueous kit (Ambion). No discernible 28S or 18S RNA species could be recovered from those reactions that did not contain BpB. In contrast, sharp 28S and 18S bands were clearly present in those reactions that did contain BpB. Enzymatic tissue digests could successfully produce intact RNA if non-denaturing RNase inhibitors, such as BpB were included.

### Example 19

### Use of the Invention to Streamline RT-PCR Analysis Without RNA Purification

Methods of the invention can be used to streamline tissue disruption and generate cDNA without formal RNA purification. This can be performed by precipitating the SDS in the tissue lysate. For example, tissue lysates were generated as described in Example 1 with frozen mouse liver. Following tissue digestion, up to 75 mM final Barium Chloride, a divalent reagent, is added to the tissue lysate to promote the spontaneous precipitation of SDS. The sample is then centrifuged, preferentially at 4 C, at 10000×g, for a minimum of 10 minutes. After centrifugation, a fraction of the supernatant is removed and added directly (no RNA purification), or diluted in nuclease-free water, to RT-PCR for quantitation and analysis. For RT-PCR, both MMLV-RT and Taq Polymerase are combined in one tube, one buffer system. GAPDH mRNA was readily detected with the direct addition of 1 ul tissue lysate into 25-ul one-step qRT-PCR, and linear detection was observed when the lysate was diluted 1:100, 1:200, and 1:1000, in nuclease-free water, prior to qRT-PCR. Thus, direct detection of transcript targets from the methods of the invention are possible without formal RNA isolation.

In a related experiment, mouse liver tissue was enzymatically digested for 20 min at 37 C with 1000 rpm mixing in a buffer containing 10 mM Tris pH 8.0, 2 mM CaCl2, 0.5 mM EDTA, and 1% Triton X-100. After liquefaction, the sample was centrifuged, and the supernatant diluted 200 to 400 times. 5 ul was added to a 25 ul qPCR using the PCR reagents from Ambion's MessageSensor RT Kit and Ambion's SuperTaq enzyme. The 1:200 diluted lysate yielded a Ct=20.98, and the 1:400, Ct=22.04, revealing a linear response for DNA detection. As a result, both RNA and DNA are released and can be readily quantified with PCR methods using the methods described herein.

### Example 20

### Non-Limiting Example of a Kit of the Present Invention

The following Table 13 included a non-limiting example of a kit of the present invention. The components in this kit are exemplary only, and it is contemplated by the inventors that the amount of each component can be decreased, increased, removed, or substituted with other ingredients and compounds that are discussed throughout this document and that are known to those of ordinary skill in the art.

TABLE 13
Kit Components for a Non-Limiting Aspect of the Invention

| # | Component Name | Content |
|---|---|---|
| 1 | Nuclease-free Water | |
| 2 | Processing Plate and Lid | |
| 3 | RNA Binding Beads | 10 mg/ml dextran magnetic beads |
| | | (1% solid) in 0.05% NaN3 |
| 4 | 1X ELT Buffer | 10 mM CHES pH 9.0 |
| | | 2 mM CaCl₂ |
| | | 0.1 mM EDTA pH 8.0 |
| | | 2% SDS |
| 5 | RNA Binding Solution | 4.75 M NaCl |
| | | 50 mM Tris-HCl pH 8.0 |
| | | 225 mM BME |
| 6 | RNA Elution Solution | 5 mM NaCl |
| | | 0.1 mM EDTA pH 8.0 |
| 7 | Elution Tubes | RNase-free 0.5 ml tubes |
| 8 | Wash Soln. Concentrate | 10 mM KCl |
| | | 2 mM Tris-HCl pH 7.0 |
| | | 0.2 mM EDTA pH 8.0 |
| | | 80% Ethanol |
| 9 | ELT Cocktail A | 20 mg/ml Proteinase K |
| | | Proteinase K Storage Buffer: |
| | | 50 mM Tris pH 8.0; |
| | | 3 mM CaCl2; 50% Glycerol |
| 10 | ELT Cocktail B | 20 mg/ml Subtilisin Carlsberg |
| | | Subtilisin Carlsberg Storage Buffer: |
| | | 50 mM Tris pH 8.0; 3 mM CaCl2; 50% |
| | | Glycerol |
| 11 | DNase I 2 U/ul | 2 U/ul in 50% Glycerol |
| 12 | 10X DNase I Buffer | 100 mM Tris pH 7.5 |
| | | 25 mM MgCₗ₂ |
| | | 5 mM CaCl₂ |

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,683,195
U.S. Pat. No. 4,683,202
U.S. Pat. No. 4,800,159
U.S. Pat. No. 4,883,750
U.S. Pat. No. 5,412,087
U.S. Pat. No. 5,445,934
U.S. Pat. No. 5,514,545
U.S. Pat. No. 5,545,522
U.S. Pat. No. 5,744,305
U.S. Pat. No. 5,843,650
U.S. Pat. No. 5,846,709
U.S. Pat. No. 5,846,783
U.S. Pat. No. 5,849,497
U.S. Pat. No. 5,849,546
U.S. Pat. No. 5,849,547
U.S. Pat. No. 5,858,652
U.S. Pat. No. 5,866,366
U.S. Pat. No. 5,912,148
U.S. Pat. No. 5,916,776
U.S. Pat. No. 5,916,779
U.S. Pat. No. 5,922,574
U.S. Pat. No. 5,928,905
U.S. Pat. No. 5,928,906
U.S. Pat. No. 5,932,451
U.S. Pat. No. 5,935,825
U.S. Pat. No. 5,939,291
U.S. Pat. No. 5,942,391
U.S. Pat. No. 6,309,823
U.S. Pat. No. 6,316,193
U.S. Pat. No. 6,322,971
U.S. Pat. No. 6,324,479
U.S. Pat. No. 6,329,140
U.S. Pat. No. 6,329,209
U.S. Patent Appln. 10/786,875
U.S. Patent Appln. 10/955,974
U.S. Patent Appln. 20030104468
U.S. Patent Appln. 60/449,912
U.S. Patent Appln. 60/547,721
PCT Appln. PCT/US87/00880
PCT Appln. PCT/US89/01025
PCT Appln. PCT/US97/07012
PCT Appln. PCT/US97/07013
PCT Appln. WO 88/10315
PCT Appln. WO 89/06700
DeRisi et al., Nature Genetics, 14:457-460, 1996.
Efstratiadis et al., Proc. Natl. Acad. Sci. USA, 73(7):2289-2293, 1976.
European Appl. 320 308
European Appl. 329 822
Fodor et al., Biochemistry, 30(33):8102-8108, 1991.
Frohman, PCR Protocols: A Guide To Methods And Applications, Academic Press, NY, 1990.
GB Appln. 2 202 328
Gubler and Hoffmann, Gene, 25:263-269, 1983.
Hacia et al., Nature Genet., 14:441-449, 1996.
Higuchi et al., Proc. Natl. Acad. Sci. USA, 73(9):3146-3150, 1976.
Innis et al., Proc. Natl. Acad. Sci. USA, 85(24):9436-9440, 1988.
Jackson et al., J. Clin. Pathol., 43(6):499-504, 1990.
Kwoh et al., Proc. Natl. Acad. Sci. USA, 86:1173, 1989.
Lai et al., Biotechniques, 15(4):620, 622, 624-626, 1993.
Land et al., Nucleic Acids Res., 9(10):2251-2266, 1981.
Maniatis et al., Cell, 8:163, 1976.
Maniatis, et al., Molecular Cloning, A Laboratory Manual, Cold Spring Harbor Press, Cold Spring Harbor, N.Y., 1990.
Maskos et al., Nucleic Acids Res., 20(7):1679-1684, 1992.
McCutcheon, In: Detergents and Emulsifiers, North America Ed., (1986), Allured Publishing Corporation, 1986.
McCutcheon, In: Functional Materials, North American Ed., Allured Publishing Corporation, 1992.
Ohara et al., Proc. Natl. Acad. Sci. USA, 86:5673-5677, 1989.
Okayama et al., Mol. Cell. Biol., 2(2):161-170, 1982.
Patanjali et al., Proc. Natl. Acad. Sci. USA, 88(5):1943-1947, 1991.
Pease et al., Proc. Natl. Acad. Sci. USA, 91:5022-5026, 1994.
Sambrook et al., In: Molecular cloning, Cold Spring Harbor Laboratory Press, Cold Spring Harbor, N.Y., 1989.
Sambrook et al., In: Molecular cloning, Cold Spring Harbor Laboratory Press, Cold Spring Harbor, N.Y., 2001.
Schena et al., Proc. Natl. Acad. Sci. USA, 93:10614-10619, 1996.
Schena, et al., Science, 270:467-470, 1995.
Shalon et al., Genome Res., 6(7):639-645, 1996.
Shoemaker et al., Nature Genetics, 14:450-456, 1996.
Walker et al., Proc. Natl. Acad. Sci. USA, 89:392-396, 1992.

What is claimed is:

1. A method comprising:
agitating an admixture at ambient temperature, the admixture comprising at least one cell-containing sample, proteinase K, subtilisin, a buffer at pH 9.0, and at least one nuclease inhibitor;
wherein the sample comprises a solid tissue sample;
wherein the nuclease inhibitor comprises 2% anionic detergent; and
wherein the sample is digested without contacting a mechanical object to produce a nucleic acid-containing lysate of the sample.

2. A kit for preserving RNA or producing a digested lysate of a tissue sample without homogenizing the sample, comprising, in a suitable container:
a buffer;
a catabolic enzyme; and
an ionic detergent.

3. A sample lysis digestion solution comprising:
at least one cell-containing sample, which comprises a cell-containing nucleic acid;
at least one a catabolic enzyme;
at least one nuclease inhibitor; and
a buffer, wherein the buffer includes a pH range of between about 7 and about 10, and wherein the buffer is formulated to maintain the activity of the catabolic enzyme and the nuclease inhibitor,
wherein the digestion of the cell-containing sample occurs without homogenizing the sample.

4. A method of preserving RNA in a tissue lysate comprising:
obtaining at least one cell-containing sample, which comprises a cell containing RNA;
obtaining at least one catabolic enzyme;
obtaining at least one ribonuclease inhibitor;
preparing an admixture of the sample, the catabolic enzyme, and the ribonuclease inhibitor;
maintaining the admixture under conditions where the catabolic enzyme is active; and;
agitating the admixture,
wherein the sample is digested to produce an RNA containing lysate in which the RNA is preserved.
